(19) United States
(12) Patent Application Publication
(10) Pub. No.: US 2005/0148646 A1
(43) Pub. Date: Jul. 7, 2005

(54) Dicationic triaryl analogs as anti-protozoan agents

(76) Inventors: David W. Boykin, Atlanta, GA (US); Richard R. Tidwell, Pittsboro, NC (US); W. David Wilson, Atlanta, GA (US); Reto Brun, Basel (CH); Mohamed A. Ismail, Talkha (EG)

Correspondence Address: JENKINS, WILSON & TAYLOR, P. A., 3100 TOWER BLVD, SUITE 1400, DURHAM, NC, 27707, US

(21) Appl. No.: 10/972,715

(22) Filed: Oct. 25, 2004

Related U.S. Application Data
(60) Provisional application No. 60/514,168, filed on Oct. 24, 2003.

(51) Int. Cl. A61K 31/4184; C07D 235/24

(52) U.S. Cl. 514/394; 548/307.4

(57) ABSTRACT

Novel dicationic, heterocyclic triaryl compounds are useful in the treatment of microbial infections, such as Trypanosoma brucei rhodesiense infection and Plasmodium falciparum infection. These compounds are accordingly useful in treating second-stage human African trypanosomiasis. Pharmaceutical formulations comprising these compounds can be used in methods of treating microbial infections.

### CROSS REFERENCE TO RELATED APPLICATIONS

This application claims the benefit of and priority to U.S. Provisional Patent Application Ser. No. 60/514,168, filed Oct. 24, 2003, the disclosure of which is incorporated herein by reference in its entirety.

### TECHNICAL FIELD

The presently disclosed subject matter relates to methods of combating microbial infections with dicationic compounds. More particularly, the presently disclosed subject matter relates to methods of combating microbial infections with heterocyclic triaryl compounds, and to the novel compounds themselves.

Abbreviations

| | |
|---|---|
| δ = | chemical shift |
| Ac = | acetyl |
| AcO = | acetoxy |
| AcOH = | acetic acid |
| Ac₂O = | acetic anhydride |
| Am = | amidine |
| AmOH = | amidoxime |
| Bu = | butyl |
| ° C. = | degrees Celsius |
| calcd = | calculated |
| cm = | centimeters |
| dec = | decomposition point |
| DIBAL = | diisobutylaluminium hydride |
| DMF = | dimethylformamide |
| DMSO = | dimethylsulfoxide |
| D₂O = | deuterium oxide |
| EtOAc = | ethyl acetate |
| EtOH = | ethanol |
| FAB = | fast atom bombardment |
| g = | grams |
| h = | hours |
| HCl = | hydrogen chloride |
| HPLC = | high-pressure liquid chromatography |
| Hz = | hertz |
| kg = | kilograms |
| KO-t-Bu = | potassium tert-butoxide |
| L.d. = | Leishmania donovani |
| M = | molar |
| Me = | methyl |
| MeO = | methoxy |
| MHz = | megahertz |
| mL = | milliliters |
| mm = | millimeters |
| mM = | millimolar |
| m.p. = | melting point |
| MS = | mass spectroscopy |
| Na₂CO_{3 =} | sodium carbonate |
| Na₂SO_{4 =} | sodium sulfate |
| NBS = | N-bromosuccinimide |
| NH₂OH.HCl = | hydroxylamine hydrochloride |
| NMR = | nuclear magnetic resonance |
| OBn = | benzyloxy |
| p = | para |
| Pd—C = | 10% palladium on carbon |
| P.f. = | Plasmodium falciparum |
| psi = | pounds per square inch |
| spp. = | species |
| T.br. = | Trypanosoma brucei rhodesiense |
| THF = | tetrahydrofuran |
| TLC = | thin-layer chromatography |
| TMS = | trimethylsilyl |
| UV = | ultraviolet |

### BACKGROUND

The incidence of microbial infections (e.g., mycobacterial, fungal, and protozoal infections) in the immunocompromised population has significantly increased over the past several years. In particular, Candida species, especially Candida albicans, are often significant pathogens in patients infected with human immunodeficiency virus (HIV). Another pathogen, Pneumocystis carinii, causes a form of pneumonia (PCP) that is believed to be one of the leading causes of death in patients suffering from AIDS. Further, Human African trypanosomiasis (HAT) has reemerged as a threat to over 60 million people. Current estimates are that between 350,000 and 450,000 people are infected. Other severe and life-threatening microbial infections are caused by Mycobacterium tuberculosis, Aspergillus spp., Cryptosporidium parvum, Giardia lamblia, Plasmodium spp., Toxoplasma gondii, Fusarium solani, and Cryptococcus neoformans.

The antimicrobial properties of dicationic molecules have been studied since the 1930's. Compounds of this type have typically utilized amidine groups as the cationic moieties, and their activities against a number of pathogens including Cryptosporidium parvum, Giardia lamblia, Leishmania spp., Plasmodium spp., Pneumocystis carini, Toxoplasma gondii, Trypanosoma spp., Candida albicans, Aspergillus spp. and Cryptococcus neoformans have been reported. See, e.g., King, H. et al., Ann. Trop. Med. Parasitol. 1938, 32, 177-192; Blagburn, B. L. et al., Antimicrob. Agents Chemother. 1991, 35, 1520-1523; Bell, C. A. et al., Antimicrob. Agents Chemother. 1991, 35, 1099-1107; Bell, C. A. et al., Antimicrob. Agents Chemother. 1990, 34, 1381-1386; Kirk, R. et al., Ann. Trop. Med. Parastiol. 1940, 34, 181-197; Fulton, J. D. Ann. Trop. Med. Parastol. 1940, 34, 53-66; Ivady, V. G. et al., Monatschr. Kinderheilkd. 1958, 106, 10-14; Boykin, D. W. et al., J. Med. Chem. 1995, 38, 912-916; Boykin, D. W. et al., J. Med. Chem. 1998, 41, 124-129; Francesconi et al., J. Med. Chem. 1999, 42, 2260-2265; Lindsay, D. S. et al., Antimicrob. Agents Chemother. 1991, 35, 1914-1916; Lourie, E. M. et al., Ann. Trop. Med. Parasitol. 1939, 33, 289-304; Lourie, E. M. et al., Ann. Trop. Med. Parasitol. 1939, 33, 305-312; Das, B. P. et al., J. Med. Chem. 1976, 20, 531-536; Del Poeta, M. et al., J. Antimicrob. Chemother. 1999, 44, 223-228; Del Poeta, M. et al., Antimicrob. Agents Chemother. 1998, 42, 2495-2502; Del Poeta, M. et al., Antimicrob. Agents Chemother. 1998, 42, 2503-2510.

Despite the broad range of activity exhibited by diamidines, only one compound of this chemical type, pentamidine, has seen significant clinical use. Pentamidine has been used clinically against African trypanosomiasis, antimony-resistant leishmaniasis, and P. carinii pneumonia. See, e.g., Apted, F. I. C., Pharmacol. Ther. 1980, 11, 391-413; Bryceson, A. D. M. et al., Trans. Roy. Soc. Trop. Med. Hyg. 1985, 79, 705-714; Hughes, W. T. et al., Antimicrob. Agents Chemother. 1974, 5, 289-293.

Thus, there is a need for compounds having antimicrobial activity, whether against the representative pathogens referenced above or against other pathogens. More particularly, there is a need for a compound having activity in the treatment of human African trypanosomiasis, an infectious disease for which oral treatment in its second stage is not currently available.

### SUMMARY

In some embodiments, the presently disclosed subject matter describes a compound comprising a diaryl ring structure of Formula (I): 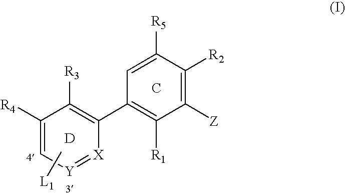
wherein:
X and Y are each independently selected from the group consisting of CH, N, O and S, and wherein Y can be present or absent;
in some embodiments, R₁, R₂, R₃, R₄ and R₅ are each independently selected from the group consisting of H, alkyl, halo, hydroxyl, alkoxyl, aryloxyl, and aralkoxyl;
in some embodiments, R₁ is selected from the group consisting of H, alkyl, halo, alkoxyl, aryloxyl, and aralkoxyl, and R₂, R₃, R₄ and R₅ are each independently selected from the group consisting of H, alkyl, halo, hydroxyl, alkoxyl, aryloxyl, and aralkoxyl;
in some embodiments, R₁, R₂, R₃, R₄ and R₅ are each independently selected from the group consisting of H, alkyl, halo, hydroxyl, alkoxyl, aryloxyl, and aralkoxyl, provided that when L₁ and L₂, as defined herein below, are both 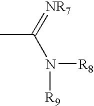
and R₇, R₈, and R₉ are each H, R₁ is not hydroxyl;
Z is selected from one of: 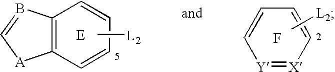
wherein:
   A is selected from the group consisting of O, S, and NR₆, and wherein R₆ is selected from one of H and alkyl;
   B is selected from the group consisting of O, S, and N;
   X′ and Y′ are each independently selected from the group consisting of CH, N, O and S, and Y′ can be present or absent;
   L₁ and L₂ are each independently selected from the group consisting of: 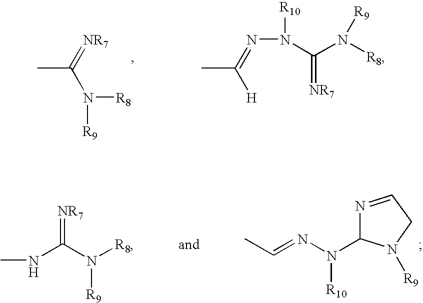
   where in:
      L₁ is at one of the 3′-position and 4′-position of the diaryl ring D;
      R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or R₇ and R₈ together are: 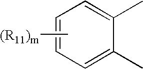
         wherein m is an integer from 1 to 3, and R₁₁ is selected from one of H and —CONHR₁₂NR₁₃R₁₄, wherein:
         R₁₂ is alkyl, and R₁₃ and R₁₄ are each independently selected from one of H and alkyl; or
            a pharmaceutically acceptable salt thereof.

In some embodiments, the presently disclosed subject matter describes a compound comprising a diaryl ring structure of Formula (II): 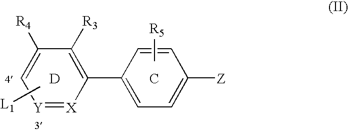
wherein:
   X and Y are each independently selected from the group consisting of CH, N, O and S, and Y can be present or absent;
   R₃, R₄, and R₅ are each independently selected from the group consisting of H, alkyl, halogen, hydroxyl, alkoxyl, aryloxyl, and aralkoxyl;
   Z is selected from one of: 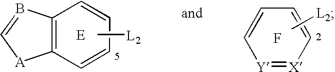
      wherein:
         A is selected from the group consisting of O, S, and NR₆, and wherein R₆ is selected from one of H and alkyl;
         B is selected from the group consisting of O, S, and N;
   X′ and Y′ are each independently selected from the group consisting of CH, N, O and S, and Y′ can be present or absent;
   L₁ and L₂ are each independently selected from the group consisting of: 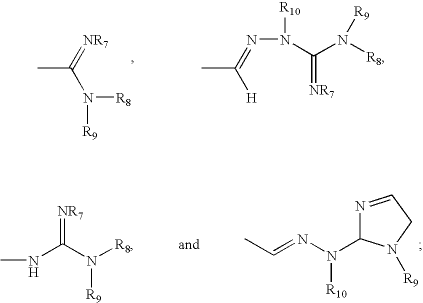
   wherein:
      L₁ is at one of the 3′-position and the 4′-position of the diaryl ring D;
      R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or
      R₇ and R₈ together are: 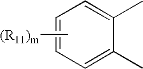
         wherein:
         m is an integer from 1 to 3, and R₁₁ is selected from one of H and —CONHR₁₂NR₁₃R₁₄, wherein R₁₂ is alkyl and R₁₃ and R₁₄ are each independently selected from one of H and alkyl; or
            a pharmaceutically acceptable salt thereof.

In some embodiments, the presently disclosed subject matter describes a pharmaceutical formulation comprising:
(a) a pharmaceutically acceptable carrier; and
(b) a compound comprising a diaryl ring structure of Formula (I): 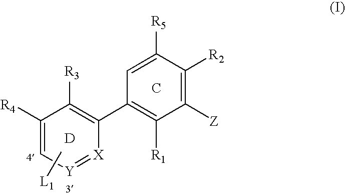
   wherein:
X and Y are each independently selected from the group consisting of CH, N, O and S, and wherein Y can be present or absent;
R₁, R₂, R₃, R₄ and R₅ are each independently selected from the group consisting of H, alkyl, halo, hydroxyl, alkoxyl, aryloxyl, and aralkoxyl;
Z is selected from one of: 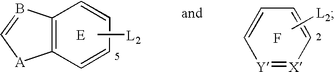
wherein:
   A is selected from the group consisting of O, S, and NR₆, and wherein R₆ is selected from one of H and alkyl;
   B is selected from the group consisting of O, S, and N;
   X′ and Y′ are each independently selected from the group consisting of CH, N, O and S, and Y′ can be present or absent;
   L₁ and L₂ are each independently selected from the group consisting of: 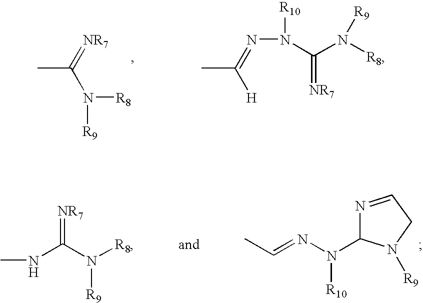
   wherein:
      L₁ is at one of the 3′-position and 4′-position of the diaryl ring D;
      R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or
      R₇ and R₈ together are: 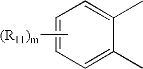
         wherein m is an integer from 1 to 3, and R₁₁ is selected from one of H and —CONHR₁₂NR₁₃R₁₄, wherein:
         R₁₂ is alkyl, and R₁₃ and R₁₄ are each independently selected from one of H and alkyl; or
            a pharmaceutically acceptable salt thereof.

In some embodiments, the presently disclosed subject matter describes a pharmaceutical formulation comprising:
(a) a pharmaceutically acceptable carrier; and
(b) a compound comprising the diaryl ring structure of Formula (II): 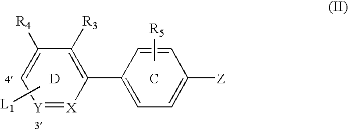
wherein:
   X and Y are each independently selected from the group consisting of CH, N, O and S, and Y can be present or absent;
   R₃, R₄, and R₅ are each independently selected from the group consisting of H, alkyl, halogen, hydroxyl, alkoxyl, aryloxyl, and aralkoxyl;
   Z is selected from one of: 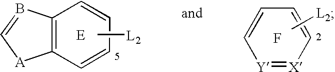
      wherein:
         A is selected from the group consisting of O, S, and NR₆, and wherein R₆ is selected from one of H and alkyl;
         B is selected from the group consisting of O, S, and N;
   X′ and Y′ are each independently selected from the group consisting of CH, N, O and S, and Y′ can be present or absent;
   L₁ and L₂ are each independently selected from the group consisting of: 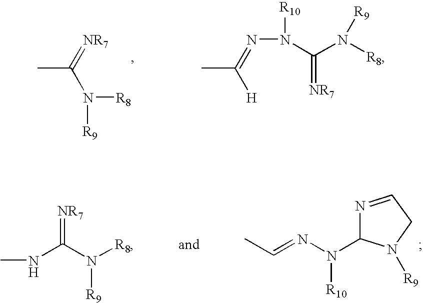
   wherein:
      L₁ is at one of the 3′-position and the 4′-position of the diaryl ring D;
      R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or
      R₇ and R₈ together are: 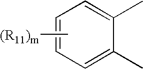
         wherein:
         m is an integer from 1 to 3, and R₁₁ is selected from one of H and —CONHR₁₂NR₁₃R₁₄, wherein R₁₂ is alkyl and R₁₃ and R₁₄ are each independently selected from one of H and alkyl; or
            a pharmaceutically acceptable salt thereof.

In some embodiments, the presently disclosed subject matter describes a method of treating a microbial infection in a subject in need thereof, the method comprising administering to the subject an effective amount of a compound comprising a diaryl ring structure of Formula (I): 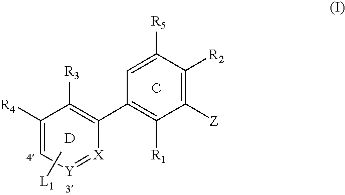
wherein:
X and Y are each independently selected from the group consisting of CH, N, O and S, and wherein Y can be present or absent;
R₁, R₂, R₃, R₄ and R₅ are each independently selected from the group consisting of H, alkyl, halo, hydroxyl, alkoxyl, aryloxyl, and aralkoxyl;
Z is selected from one of: 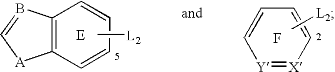
wherein:
   A is selected from the group consisting of O, S, and NR₆, and wherein R₆ is selected from one of H and alkyl;
   B is selected from the group consisting of O, S, and N;
   X′ and Y′ are each independently selected from the group consisting of CH, N, O and S, and Y′ can be present or absent;
   L₁ and L₂ are each independently selected from the group consisting of: 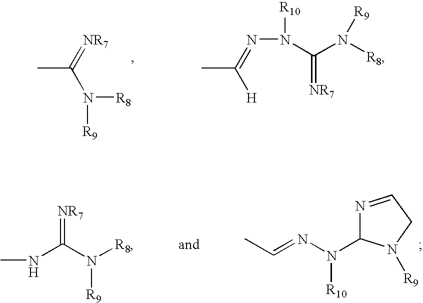
   wherein:
      L₁ is at one of the 3′-position and 4′-position of the diaryl ring D;
      R₇ is selected from the group consisting of H, alkyl hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or
      R₇ and R₈ together are: 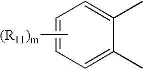
         wherein m is an integer from 1 to 3, and R₁₁ is selected from one of H and —CONHR₁₂NR₁₃R₁₄, wherein:
         R₁₂ is alkyl, and R₁₃ and R₁₄ are each independently selected from one of H and alkyl; or
            a pharmaceutically acceptable salt thereof.

In some embodiments, the presently disclosed subject matter describes a method of treating microbial infection in a subject in need thereof, the method comprising administering to the subject an effective amount of a compound comprising a diaryl ring structure of Formula (II): 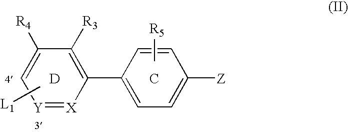
wherein:
   X and Y are each independently selected from the group consisting of CH, N, O and S, and Y can be present or absent;
   R₃, R₄, and R₅ are each independently selected from the group consisting of H, alkyl, halogen, hydroxyl, alkoxyl, aryloxyl, and aralkoxyl;
   Z is selected from one of: 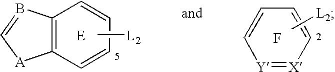
      wherein:
         A is selected from the group consisting of O, S, and NR₆, and wherein R₆ is selected from one of H and alkyl;
         B is selected from the group consisting of O, S, and N;
   X′ and Y′ are each independently selected from the group consisting of CH, N, O and S, and Y′ can be present or absent;
   L₁ and L₂ are each independently selected from the group consisting of: 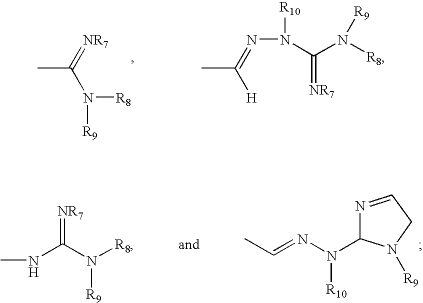
   wherein:
      L₁ is at one of the 3′-position and the 4′-position of the diaryl ring D;
      R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or
      R₇ and R₈ together are: 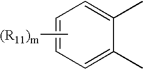
         wherein:
         m is an integer from 1 to 3, and R₁₁ is selected from one of H and —CONHR₁₂NR₁₃R₁₄, wherein R₁₂ is alkyl and R₁₃ and R₁₄ are each independently selected from one of H and alkyl; or
            a pharmaceutically acceptable salt thereof.

In some embodiments, the microbial infection is selected from the group consisting of a Trypanosoma species, Pneumocytsis carnii, Giardia lamblia, Cryptosporidium parvum, Cryptococcus neoformans, Candida albicans, Candida tropicalis, Salmonella typhimurium, Plasmodium falciparum, Leishmania donovani, and Leishmania mexicana amazonensis. In some embodiments, the Trypanosoma species comprises Trypanosoma brucei rhodesiense. In some embodiments, the microbial infection comprises a Plasmodium falciparum infection.

In some embodiments, the presently disclosed subject matter describes the use of an active compound of Formula (I) for the preparation of a medicament for treating a microbial infection. In some embodiments, the presently disclosed subject matter describes the use of an active compound of Formula (II) for the preparation of a medicament for treating a microbial infection.

Certain objects of the presently disclosed subject matter having been stated hereinabove, which are addressed in whole or in part by the presently disclosed subject matter, other aspects and objects will become evident as the description proceeds when taken in connection with the accompanying Examples as best described herein below.

### DETAILED DESCRIPTION

The presently disclosed subject matter will be now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers as well as racemic mixtures where such isomers and mixtures exist.

I. Definitions

As used herein the term “alkyl” refers to C₁₋₂₀ inclusive, linear (i.e., “straight-chain”), branched, or cyclic, saturated or unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8, i.e., 1, 2, 3, 4, 5, 6, 7 or 8, carbon atoms (i.e., a C₁₋₈ alkyl). “Higher alkyl” refers to an alkyl group having about 10 to about 20, i.e., 10, 11, 12, 13, 14, 15, 16, 17, 18, 19, or 20, carbon atoms (i.e., a C₁₀₋₂₀ alkyl). In certain embodiments, “alkyl” refers, in particular, to C₁₋₈ straight-chain alkyls. In other embodiments, alkyl refers, in particular, to C₁₋₈ branched-chain alkyls.

Alkyl groups can be optionally substituted with one or more alkyl group substituents, which can be the same or different. The term “alkyl group substituent” includes but is not limited to alkyl, halo, arylamino, acyl, hydroxyl, aryloxy, alkoxyl, alkylthio, arylthio, aralkyloxyl, aralkylthio, carboxyl, alkoxycarbonyl, oxo and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

The term “aryl” is used herein to refer to an aromatic substituent which can be a single aromatic ring, or multiple aromatic rings that are fused together, linked covalently, or linked to a common group such as a methylene or ethylene moiety. The common linking group also can be a carbonyl as in benzophenone or oxygen as in diphenylether or nitrogen as in diphenylamine. In some embodiments, the “aryl” group comprises two aromatic rings that are linked covalently and are referred to herein as a “diaryl” group or a “diaryl” ring structure or a “diaryl” compound. Examples of a diaryl group include biphenyl. Further, in some embodiments, the “aryl” group comprises three aromatic rings that are linked covalently and are referred to herein as a “triaryl” group or a “triaryl” compound.

The term “aryl” specifically encompasses heterocyclic aromatic compounds. The aromatic ring(s) can comprise phenyl, naphthyl, biphenyl, diphenylether, diphenylamine and benzophenone ring structures, among others. In particular embodiments, the term “aryl” means a cyclic aromatic comprising from about 5 to about 10, i.e., 5, 6, 7, 8, 9, or 10, carbon atoms, including 5- and 6-membered hydrocarbon and heterocyclic aromatic rings.

The aryl group can be optionally substituted with one or more aryl group substituents which can be the same or different, wherein “aryl group substituent” includes alkyl, aryl, aralkyl, hydroxyl, alkoxyl, aryloxy, aralkoxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene and —NR′R″, wherein R′ and R″ can be each independently hydrogen, alkyl, aryl and aralkyl.

As defined herein, an aryl group substituent, e.g., an “R” group, can be represented as: 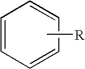
wherein the “R” group can be present at any available positions on the aromatic ring.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole and the like.

Thus, as used herein, the terms “substituted alkyl” and “substituted aryl” include alkyl and aryl groups, as defined herein, in which one or more atoms or functional groups of the aryl or alkyl group are replaced with another atom or functional group, including for example, halogen, aryl, alkyl, alkoxyl, hydroxyl, nitro, amino, alkylamino, dialkylamino, sulfate, and mercapto.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups. Specific examples of acyl groups include acetyl and benzoyl.

“Cyclic” and “cycloalkyl” refer to a non-aromatic mono- or multicyclic ring system of about 3 to about 10, i.e., 3, 4, 5, 6, 7, 8, 9, or 10, carbon atoms. The cycloalkyl group can be optionally partially unsaturated. The cycloalkyl group also can be optionally substituted with an alkyl group substituent as defined herein, oxo and/or alkylene. There can be optionally inserted along the cyclic alkyl chain one or more oxygen, sulfur or substituted or unsubstituted nitrogen atoms, wherein the nitrogen substituent is hydrogen, lower alkyl, or aryl, thus providing a heterocyclic group. Representative monocyclic cycloalkyl rings include cyclopentyl, cyclohexyl and cycloheptyl. Multicyclic cycloalkyl rings include adamantyl, octahydronaphthyl, decalin, camphor, camphane, and noradamantyl.

“Alkoxyl” or “alkoxyalkyl” refer to an alkyl-O— group wherein alkyl is as previously described herein. The term “alkoxyl” as used herein can refer to C₁₋₂₀ inclusive, linear, branched, or cyclic, saturated or unsaturated oxo-hydrocarbon chains, including, for example, methoxy, ethoxy, propoxy, isopropoxy, butoxy, t-butoxy, and pentoxy.

“Aryloxyl” refers to an aryl-O— group wherein the aryl group is as previously described herein. The term “aryloxyl” as used herein can refer to phenyloxyl or hexyloxyl, and alkyl, halo, or alkoxyl substituted phenyloxyl or hexyloxyl.

“Aralkyl” refers to an aryl-alkyl-group wherein aryl and alkyl are as previously described herein. Exemplary aralkyl groups include benzyl, phenylethyl and naphthylmethyl.

“Aralkyloxyl” refers to an aralkyl-O— group wherein the aralkyl group is as previously described herein. An exemplary aralkyloxy group is benzyloxy.

“Dialkylamino” refers to an —NRR′ group wherein each of R and R′ is independently an alkyl group as previously described herein. Exemplary alkylamino groups include ethylmethylamino, dimethylamino and diethylamino.

“Alkoxycarbonyl” refers to an alkyl-O—CO— group. Exemplary alkoxycarbonyl groups include methoxycarbonyl, ethoxycarbonyl, butyloxycarbonyl and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl as previously described herein.

“Dialkylcarbamoyl” refers to R′RN—CO— group wherein each of R and R′ is independently alkyl as previously described herein.

“Acyloxyl” refers to an acyl-O— group wherein acyl is as previously described herein.

“Acylamino” refers to an acyl-NH— group wherein acyl is as previously described herein.

“Aroylamino” refers to an aroyl-NH— group wherein aroyl is as previously described herein.

“Alkylene” refers to a straight or branched bivalent aliphatic hydrocarbon group having from 1 to about 20, i.e., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulphur or substituted or unsubstituted nitrogen atoms (also referred to herein as “alkylaminoalkyl”), wherein the nitrogen substituent is alkyl as previously described herein. Exemplary alkylene groups include methylene (—CH₂—); ethylene (—CH₂—CH₂—); propylene (—(CH₂)₃—); cyclohexylene (—C₆H₁₀—); —CH═CH—CH═C H—; —CH═CH—CH₂; —(CH₂)ₙ—N(R)—(CH₂)ₘ—, wherein each of m and n is independently an integer from 0 to about 20, i.e., 0, 1, 2, 3, 4, 5, 6, 7, 8, 9, 10, 11, 12, 13, 14, 15, 16, 17, 18, 19, or 20 and R is hydrogen or lower alkyl; methylenedioxy (—O—CH₂—O—); and ethylenedioxy (—O—(CH₂)₂—O—). An alkylene group can have about 2 to about 3 carbon atoms and can further have from about 6 to about 20 carbons.

The term “amino” refers to the —NH₂ group.

The term “carbonyl” refers to the —(C═O)— group.

The term “carboxyl” refers to the —COOH group.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄ group.

When the term “independently selected” is used, the substituents being referred (i.e., R groups, such as groups R₁, and R₂, or groups X and Y), can be identical or different. For example, R₂ and R₃ may both be substituted alkyls, or R₂ may be hydrogen and R₃ may be a substituted aryl, and the like.

A named “R”, “X,” “X′,” “Y,” “Y′,” “A,” “B,” “L,” or “Z” group generally will have the structure that is recognized in the art as corresponding to a group having that name, unless specified otherwise herein. For the purposes of illustration, certain representative “R,” “X,” “Y” groups as set forth above are defined below. These definitions are intended to supplement and illustrate, not preclude, the definitions known to those of skill in the art.

II. Novel Compounds

A. Compounds of Formula (I)

Described herein is a compound comprising the diaryl ring structure of Formula (I): 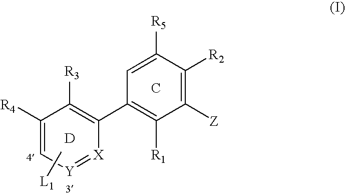
wherein:
X and Y are each independently selected from the group consisting of CH, N, O and S, and wherein Y can be present or absent;
in some embodiments, R₁, R₂, R₃, R₄ and R₅ are each independently selected from the group consisting of H, alkyl, halo, hydroxyl, alkoxyl, aryloxyl, and aralkoxyl;
in some embodiments, R₁ is selected from the group consisting of H, alkyl, halo, alkoxyl, aryloxyl, and aralkoxyl, and R₂, R₃, R₄ and R₅ are each independently selected from the group consisting of H, alkyl, halo, hydroxyl, alkoxyl, aryloxyl, and aralkoxyl;
in some embodiments, R₁, R₂, R₃, R₄ and R₅ are each independently selected from the group consisting of H, alkyl, halo, hydroxyl, alkoxyl, aryloxyl, and aralkoxyl, provided that when L₁ and L₂, as defined herein below, are both 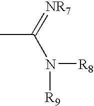
and R₇, R₈, and R₉ are each H, R₁ is not hydroxyl;
Z is selected from one of: 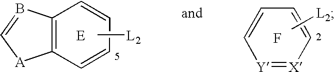
wherein:
   A is selected from the group consisting of O, S, and NR₆, and wherein R₆ is selected from one of H and alkyl;
   B is selected from the group consisting of O, S, and N;
   X′ and Y′ are each independently selected from the group consisting of CH, N, O and S, and Y′ can be present or absent;
   L₁ and L₂ are each independently selected from the group consisting of: 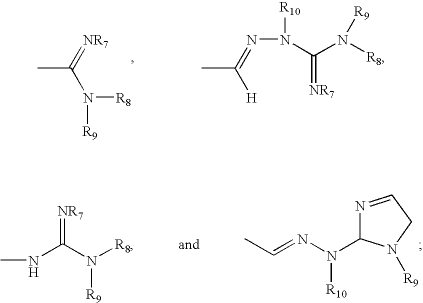
   wherein:
      L₁ is at one of the 3′-position and 4′-position of the diaryl ring D;
      R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or
      R₇ and R₈ together are: 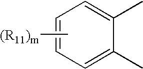
         wherein m is an integer from 1 to 3, and R₁₁ is selected from one of H and —CONHR₁₂NR₁₃R₁₄, wherein:
         R₁₂ is alkyl, and R₁₃ and R₁₄ are each independently selected from one of H and alkyl; or
            a pharmaceutically acceptable salt thereof.

In some embodiments, X is selected from one of CH and N; Y is present and is CH; Z is 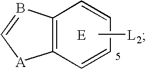
wherein A is NH; B is N; and L₂ is at the 5-position of ring E; L₁ and L₂ are each independently 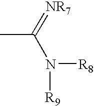
wherein R₇ is selected from one of H and hydroxyl; and R₈ and R₉ are each H; R₁ and R₄ are each H; R₂ is selected from the group consisting of H, hydroxyl, and alkoxyl; R₃ is selected from one of H and alkyl; and R₅ is selected from one of H and alkoxyl.

In some embodiments, X is CH; R₂ is selected from the group consisting of H, hydroxyl, and alkoxyl; R₃ is selected from one of H and alkyl; R₅ is selected from one of H and alkoxyl; and R₇ is selected from one of H and hydroxyl.

In some embodiments, R₂, R₃, and R₅ are each H. In some embodiments, R₂ is hydroxyl. In some embodiments, at least one of R₂ and R₅ is alkoxyl. In some embodiments, R₃ is alkyl. In some embodiments, R₇ is H. In some embodiments, R₇ is hydroxyl. In some embodiments, L₁ is at the 4′-position of the diaryl ring D. In some embodiments, L₁ is at the 3′-position of the diaryl ring D.

In some embodiments, X is N; R₃ and R₅ are each H; R₂ is selected from one of H and alkoxyl; and R₇ is selected from one of H and hydroxyl. In some embodiments, R₂ is H. In some embodiments, R₂ is alkoxyl. In some embodiments, R₇ is H. In some embodiments, R₇ is OH. In some embodiments, L₁ is in the 4′-position of the diaryl ring D.

In some embodiments, X is 0; Y is absent; Z is 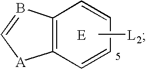
wherein A is NH; B is N; and L₂ is at the 5-position of ring E; L₁ and L₂ are each independently 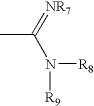
wherein R₇ is selected from one of H and hydroxyl; and R₈ and R₉ are each H; and R₁, R₂, R₃, R₄, and R₅ are each H. In some embodiments, R₇ is H.

Representative compounds of Formula (I) include, but are not limited to: N-hydroxy-2-[4-hydroxy-4′-(N-hydroxycarbamimidoyl)-biphenyl-3-yl]-1H-benzimidazole-5-carboxamidine (3); 2-(4′-carbamimidoyl-4-hydroxy-biphenyl-3-yl)-1H-benzimidazole-5-carboxamidine (4); N-hydroxy-2-[4-hydroxy-3′-(N-hydroxycarbamimidoyl)-biphenyl-3-yl]-1H-benzimidazole-5-carboxamidine (7); 2-(3′-carbamimidoyl-4-hydroxy-biphenyl-3-yl)-1H-benzimidazole-5-carboxamidine (8); N-hydroxy-2-[4-hydroxy-4′-(N-hydroxycarbamimidoyl)-5-methoxy-biphenyl-3-yl] 1H-benzimidazole-5-carboxamidine (11); 2-(4′-carbamimidoyl-4-hydroxy-5-methoxy-biphenyl-3-yl)-1H-benzimidazole-5-carboxamidine (12); 2-(4′-carbamimidoyl-2-hydroxy-biphenyl-3-yl)-1H-benzimidazole-5-carboxamidine (16); N-hydroxy-2-[4′-(N-hydroxycarbamimidoyl)-4-methoxy-biphenyl-3-yl]-1H-benzimidazole-5-carboxamidine (19); 2-(4′-carbamimidoyl-4-methoxy-biphenyl-3-yl)-1H-benzimidazole-5-carboxamidine (20); N-hydroxy-2-[4′-(N-hydroxycarbamimidoyl)-biphenyl-3-yl]-1H-benzimidazole-5-carboxamidine (23); 2-(4′-carbamimidoyl-biphenyl-3-yl)-1H-benzimidazole-5-carboxamidine (24); N-hydroxy-2-[4′-(N-hydroxycarbamimidoyl)-2′-methyl-biphenyl-3-yl]-1H-benzimidazole-5-carboxamidine (27); 2-(4′-carbamimidoyl-2′-methyl-biphenyl-3-yl)-1H-benzimidazole-5-carboxamidine (28); N-hydroxy-2-{5-[5-(N-hydroxycarbamimidloyl)-pyridin-2-yl]-2-methoxy-phenyl}-1H-benzimidazole-5-carboxamidine (35); 2-[5-(5-carbamimidoyl-pyridin-2-yl)-2-methoxyphenyl]-1H-benzimidazole-5-carboxamidine (36); N-hydroxy-2-{3-[5-(N-hydroxycarbamimidoyl)-pyridin-2-yl]-phenyl}-1H-benzimidazole-5-carboxamidine (39); and 2-[3-(5-Carbamimidoyl-pyridin-2-yl)-phenyl]-1H-benzimidazole-5-carboxamidine (40).

In some embodiments, the compound of Formula (I) has the following structure:

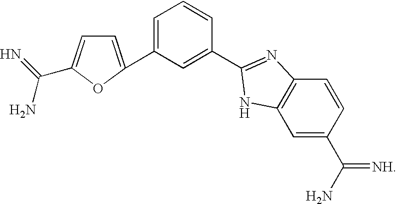

B. Compounds of Formula (II)

Described herein is a compound comprising the diaryl ring structure of Formula (II): 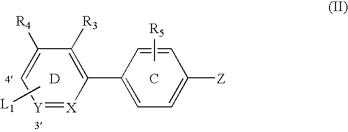
wherein:
   X and Y are each independently selected from the group consisting of CH, N, O and S, and Y can be present or absent;
   R₃, R₄, and R₅ are each independently selected from the group consisting of H, alkyl, halogen, hydroxyl, alkoxyl, aryloxyl, and aralkoxyl;
   Z is selected from one of: 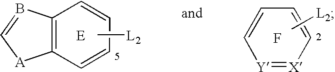
      wherein:
         A is selected from the group consisting of O, S, and NR₆, and wherein R₆ is selected from one of H and alkyl;
         B is selected from the group consisting of O, S, and N;
   X′ and Y′ are each independently selected from the group consisting of CH, N, O and S, and Y′ can be present or absent;
   L₁ and L₂ are each independently selected from the group consisting of: 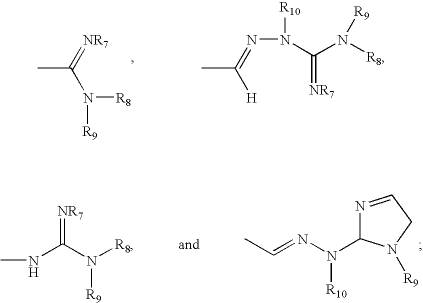
   wherein:
      L₁ is at one of the 3′-position and the 4′-position of the diaryl ring D;
      R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or
      R₇ and R₈ together are: 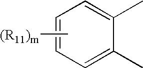
         wherein:
         m is an integer from 1 to 3, and R₁₁ is selected from one of H and —CONHR₁₂NR₁₃R₁₄, wherein R₁₂ is alkyl and R₁₃ and R₁₄ are each independently selected from one of H and alkyl; or
            a pharmaceutically acceptable salt thereof.

In some embodiments, X and Y are each CH; R₃ is selected from the group consisting of H, alkyl, hydroxyl, alkoxyl, and araloxyl; R₄ is selected from one of H and halogen; R₅ is H; Z is: 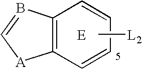
wherein A is NH; B is N; L₁ and L₂ are each independently: 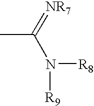
wherein R₇ is selected from one of H and hydroxyl; and R₈ and R₉ are each H. In some embodiments, R₃ is H. In some embodiments, R₃ is alkyl. In some embodiments, R₃ is hydroxyl. In some embodiments, R₃ is araloxyl. In some embodiments, R₄ is H. In some embodiments, R₄ is halogen. In some embodiments, R₇ is H. In some embodiments, R₇ is hydroxyl.

In some embodiments, X is N; Y is CH; R₃, R₄, and R₅ are each H; Z is: 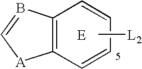
wherein A is NH; B is N; L₁ and L₂ are each independently: 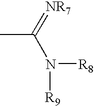
wherein L₁ is in the 4′-position of the diaryl ring D; R₇ is selected from one of H and hydroxyl; and R₈ and R₉ are each H. In some embodiments, R₇ is H. In some embodiments, R₇ is OH.

In some embodiments, X and Y are each CH; R₃, R₄, and R₅ are each H; Z is: 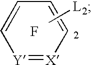
wherein X′ is O; Y′ is absent; L₁ and L₂ are each independently selected from the group consisting of: 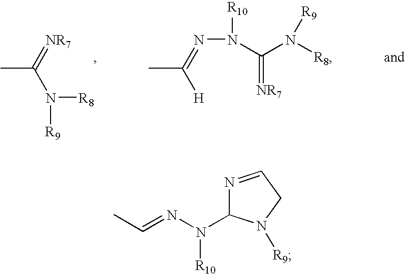
wherein R₇ is selected from one of H and OH; and R₈, R₉ and R₁₀ are H. In some embodiments, L₁ and L₂ are each independently:

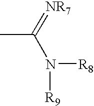

In some embodiments, R₇ is H. In some embodiments, R₇ is OH. In some embodiments, L₁ and L₂ are each independently: 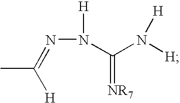
and R₇ is H.

In some embodiments, L₁ and L₂ are each independently:

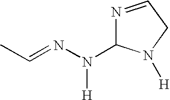

Representative compounds of Formula (II) include, but are not limited to: 2-[3-fluoro-4′-(N-hydroxycarbamimidoyl)-biphenyl-4-yl]-N-hydroxy-1H-benzimidazole-5-carboxamidine (31); 2-(4′-carbamimidoyl-3′-fluoro-biphenyl-4-yl)-1H-benzimidazole-5-carboxamidine (32); N-hydroxy-2-{4-[5-(N-hydroxycarbamimidoyl)-pyridin-2-yl]-phenyl}-1H-benzimidazole-5-carboxamidine (43); 2-[4-(5-carbamimidoyl-pyridin-2-yl)-phenyl]-1H-benzimidazole-5-carboxamidine (44); 2-[2′-benzyloxy-4′-(N-hydroxycarbamimidoyl)-biphenyl-4-yl]-N-hydroxy-1H-benzimidazole-5-carboxamidine (47); 2-(4′-carbamimidoyl-2′-hydroxy-biphenyl-4-yl)-1H-benzimidazole-5-carboxamidine (48); N-hydroxy-2-[4′-(N-hydroxycarbamimidoyl)-2′-methyl-biphenyl-4-yl]-1H-benzimidazole-5-carboxamidine (51); and 2-(4′-carbamimidoyl-2′-methylbiphenyl-4-yl)-1H-benzimidazole-5-carboxamidine (52).

In some embodiments, the compound of Formula (II) is selected from the group of compounds having the following chemical structures:

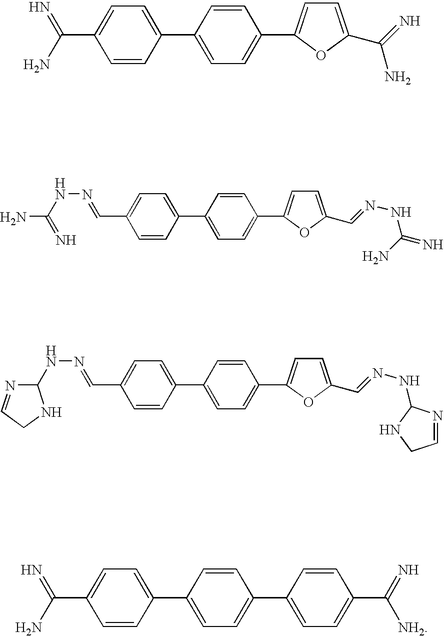

C. Prodrugs

In some embodiments, compounds disclosed herein are prodrugs. A prodrug means a compound that, upon administration to a recipient, is capable of providing (directly or indirectly) a compound of the presently disclosed subject matter or an inhibitorily active metabolite or residue thereof. Prodrugs can increase the bioavailability of the compounds of the presently disclosed subject matter when such compounds are administered to a subject (e.g., by allowing an orally administered compound to be more readily absorbed into the blood) or can enhance delivery of the parent compound to a biological compartment (e.g., the brain or lymphatic system) relative to a metabolite species, for example. Compounds 3, 7, 11, 15, 19, 20, 27, 31, 43, 47, and 51 described in Examples 1-5,7-8, and 11-13 of the presently disclosed subject matter are prodrugs.

D. Pharmaceutically Acceptable Salts

Additionally, the active compounds of the presently disclosed subject matter can be administered as pharmaceutically acceptable salts. Such salts include the gluconate, lactate, acetate, tartarate, citrate, phosphate, borate, nitrate, sulfate, and hydrochloride salts. The salts of the compounds described herein can be prepared, in general, by reacting two equivalents of the base compound with the desired acid, in solution. After the reaction is complete, the salts are crystallized from solution by the addition of an appropriate amount of solvent in which the salt is insoluble. In some embodiments, the pharmaceutically acceptable salt is an acetate salt.

III. Pharmaceutical Formulations

The compounds of Formula (I) and Formula (II), the pharmaceutically acceptable salts thereof, prodrugs corresponding to compounds of Formula (I) and Formula (II), and the pharmaceutically acceptable salts thereof, are all referred to herein as “active compounds.” Pharmaceutical formulations comprising the aforementioned active compounds also are provided herein. These pharmaceutical formulations comprise active compounds as described herein, in a pharmaceutically acceptable carrier.

With regard to the presently described pharmaceutical formulation embodiments, compounds of Formula (I) are defined as having a structure as follows: 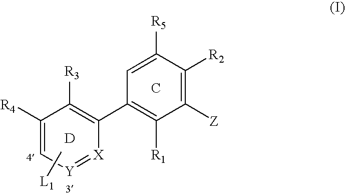
wherein:
X and Y are each independently selected from the group consisting of CH, N, O and S, and wherein Y can be present or absent;
R₁, R₂, R₃, R₄ and R₅ are each independently selected from the group consisting of H, alkyl, halo, hydroxyl, alkoxyl, aryloxyl, and aralkoxyl;
Z is selected from one of: 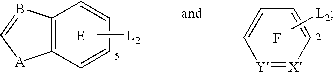
wherein:
   A is selected from the group consisting of O, S, and NR₆, and wherein R₆ is selected from one of H and alkyl;
   B is selected from the group consisting of O, S, and N;
   X′ and Y′ are each independently selected from the group consisting of CH, N, O and S, and Y′ can be present or absent;
   L₁ and L₂ are each independently selected from the group consisting of: 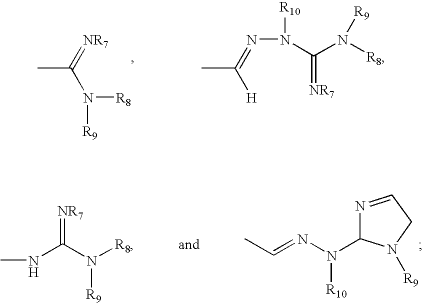
   wherein:
      L₁ is at one of the 3′-position and 4′-position of the diaryl ring D;
      R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or
      R₇ and R₈ together are: 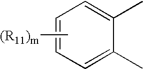
         wherein m is an integer from 1 to 3, and R₁₁ is selected from one of H and —CONHR₁₂NR₁₃R₁₄, wherein:
         R₁₂ is alkyl, and R₁₃ and R₁₄ are each independently selected from one of H and alkyl; or
            a pharmaceutically acceptable salt thereof.

Further, with regard to the presently described pharmaceutical formulation embodiments, compounds of Formula (II) are defined as having a structure as follows: 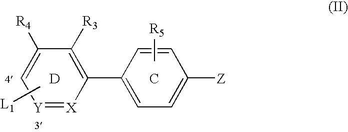
wherein:
   X and Y are each independently selected from the group consisting of CH, N, O and S, and Y can be present or absent;
   R₃, R₄, and R₅ are each independently selected from the group consisting of H, alkyl, halogen, hydroxyl, alkoxyl, aryloxyl, and aralkoxyl;
   Z is selected from one of: 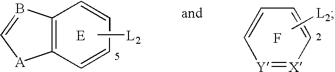
      wherein:
         A is selected from the group consisting of O, S, and NR₆, and wherein R₆ is selected from one of H and alkyl;
         B is selected from the group consisting of O, S, and N;
   X′ and Y′ are each independently selected from the group consisting of CH, N, O and S, and Y′ can be present or absent;
   L₁ and L₂ are each independently selected from the group consisting of: 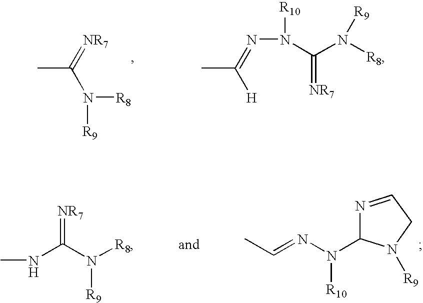
   wherein:
      L₁ is at one of the 3′-position and the 4′-position of the diaryl ring D;
      R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or
      R₇ and R₈ together are: 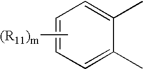
         wherein:
         m is an integer from 1 to 3, and R₁₁ is selected from one of H and —CONHR₁₂NR₁₃R₁₄, wherein R₁₂ is alkyl and R₁₃ and R₁₄ are each independently selected from one of H and alkyl; or
            a pharmaceutically acceptable salt thereof.

Pharmaceutical formulations can be prepared for oral, intravenous, or aerosol administration as described in greater detail herein below. Also, the presently disclosed subject matter provides such active compounds that have been lyophilized and that can be reconstituted to form pharmaceutically acceptable formulations for administration, as by intravenous or intramuscular injection.

The therapeutically effective dosage of any specific active compound, the use of which is in the scope of embodiments described herein, will vary somewhat from compound to compound, and patient to patient, and will depend upon the condition of the patient and the route of delivery. As a general proposition, a dosage from about 0.1 mg/kg to about 50 mg/kg will have therapeutic efficacy, with all weights being calculated based upon the weight of the active compound, including the cases where a salt is employed. Toxicity concerns at the higher level can restrict intravenous dosages to a lower level such as up to about 10 mg/kg, with all weights being calculated based upon the weight of the active base, including the cases where a salt is employed. A dosage from about 10 mg/kg to about 50 mg/kg can be employed for oral administration. Typically, a dosage from about 0.5 mg/kg to 5 mg/kg can be employed for intramuscular injection. In some embodiments, dosages range from about 1 μmol/kg to about 50 μmol/kg. In some embodiments, dosages range from about 22 μmol/kg to about 33 μmol/kg of the compound for intravenous or oral administration. The duration of the treatment typically is once per day for a period of two to three weeks or until the condition is essentially controlled. Lower doses given less frequently can be used prophylactically to prevent or reduce the incidence of recurrence of the infection.

In accordance with the presently disclosed methods, pharmaceutically active compounds as described herein can be administered orally as a solid or as a liquid, or can be administered intramuscularly or intravenously as a solution, suspension, or emulsion. Alternatively, the compounds or salts can be administered by inhalation, intravenously or intramuscularly as a liposomal suspension. When administered through inhalation the active compound or salt should be in the form of a plurality of solid particles or droplets having, in some embodiments, a particle size from about 0.5 to about 5 microns, and in some embodiments, a particle size from about 1 to about 2 microns.

Pharmaceutical formulations suitable for intravenous or intramuscular injection are provided herein. The pharmaceutical formulations comprise a compound of Formula (I) or Formula (II) described herein, a prodrug as described herein, or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. Of course, the dispensing is preferably done by an aseptic method. Sterilized closures can then be placed on the vials and, if desired, the vial contents may be lyophilized.

In addition to compounds of Formula (I) and Formula (II) or their salts or prodrugs, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. The antimicrobial preservative is typically employed when the formulation is placed in a vial designed for multi-dose use. The pharmaceutical formulations described herein can be lyophilized using techniques well known in the art.

In some embodiments of the subject matter described herein, there is provided an injectable, stable, sterile formulation comprising a compound of any one of Formula (I) and Formula (II), or a salt thereof, in a unit dosage form in a sealed container. The compound or salt is provided in the form of a lyophilizate, which is capable of being reconstituted with a suitable pharmaceutically acceptable carrier to form a liquid formulation suitable for injection thereof into a subject. The unit dosage form typically comprises from about 10 mg to about 10 grams of the compound salt. When the compound or salt is substantially water-insoluble, a sufficient amount of emulsifying agent, which is physiologically acceptable, can be employed in sufficient quantity to emulsify the compound or salt in an aqueous carrier. One such useful emulsifying agent is phosphatidyl choline.

Other pharmaceutical formulations can be prepared from the water-insoluble compounds disclosed herein, or salts thereof, such as aqueous base emulsions. In such an instance, the formulation will contain a sufficient amount of pharmaceutically acceptable emulsifying agent to emulsify the desired amount of the compound or salt thereof. Particularly useful emulsifying agents include phosphatidyl cholines and lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound will be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques.

The liposomal formulations containing the active compounds disclosed herein can be lyophilized to produce a lyophilizate, which can be reconstituted with a pharmaceutically acceptable carrier, such as water, to regenerate a liposomal suspension.

Pharmaceutical formulations also are provided which are suitable for administration as an aerosol, by inhalation. These formulations comprise a solution or suspension of a desired compound described herein or a salt thereof, or a plurality of solid particles of the compound or salt. The desired formulation can be placed in a small chamber and nebulized. Nebulization can be accomplished by compressed air or by ultrasonic energy to form a plurality of liquid droplets or solid particles comprising the compounds or salts. In some embodiments, the liquid droplets or solid particles have a particle size in the range of about 0.5 microns to about 10 microns, in some embodiments, the liquid droplets or solid particles have a particle size in the range from about 0.5 microns to about 5 microns. The solid particles can be obtained by processing the solid compound or a salt thereof, in any appropriate manner known in the art, such as by micronization. In some embodiments, the size of the solid particles or droplets will be from about 1 micron to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation will comprise a water-soluble active compound in a carrier that comprises water. A surfactant can be present, which lowers the surface tension of the formulation sufficiently to result in the formation of droplets within the desired size range when subjected to nebulization.

As indicated, both water-soluble and water-insoluble active compounds are provided. As used in the presently disclosed subject matter, the term “water-soluble” is meant to define any composition that is soluble in water in an amount of about 50 mg/mL, or greater. Also, as used in the presently described subject matter, the term “water-insoluble” is meant to define any composition that has solubility in water of less than about 20 mg/mL. For certain applications, water-soluble compounds or salts can be desirable whereas for other applications water-insoluble compounds or salts likewise can be desirable.

IV. Methods of Treating Microbial Infections

Subjects with microbial infections can be treated by methods described herein. These infections can be caused by a variety of microbes, including fungi, algae, protozoa, bacteria, and viruses. Exemplary microbial infections that can be treated by the method of the presently disclosed subject matter include, but are not limited to, infections caused by Trypanosoma species (e.g., Trypanosoma brucei rhodesiense), Pneumocytsis carnii, Giardia lamblia, Cryptosporidium parvum, Cryptococcus neoformans, Candida albicans, Candida tropicalis, Salmonella typhimurium, Plasmodium falciparum, Leishmania donovani, and Leishmania mexicana amazonensis. In some embodiments, the microbial infection is selected from one of Trypanosoma brucei rhodesiense and Plasmodium falciparum. In some embodiments, the microbial infection comprises Trypanosoma brucei rhodesiense. In some embodiments, the microbial infection comprises Plasmodium falciparum. The methods of the presently disclosed subject matter are useful for treating these conditions in that they inhibit the onset, growth, or spread of the condition, cause regression of the condition, cure the condition, or otherwise improve the general well-being of a subject afflicted with, or at risk of contracting the condition.

Methods of treating microbial infections comprise administering to a subject in need of treatment an active compound as described herein. These active compounds, as set forth above, include compounds of Formula (I) and Formula (II), their corresponding prodrugs, and pharmaceutically acceptable salts of the compounds and prodrugs. With regard to the presently described method embodiments, compounds of Formula (I) are defined as having a structure as follows: 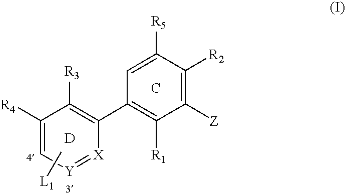
wherein:
X and Y are each independently selected from the group consisting of CH, N, O and S, and wherein Y can be present or absent;
R₁, R₂, R₃, R₄ and R₅ are each independently selected from the group consisting of H, alkyl, halo, hydroxyl, alkoxyl, aryloxyl, and aralkoxyl;
Z is selected from one of: 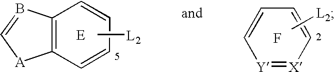
wherein:
   A is selected from the group consisting of O, S, and NR₆, and wherein R₆ is selected from one of H and alkyl;
   B is selected from the group consisting of O, S, and N;
   X′ and Y′ are each independently selected from the group consisting of CH, N, O and S, and Y′ can be present or absent;
   L₁ and L₂ are each independently selected from the group consisting of: 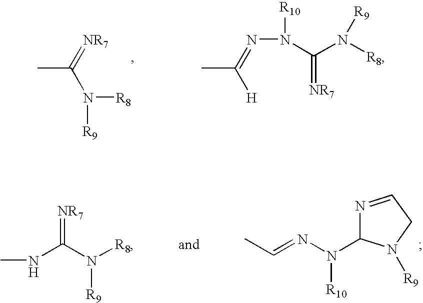
   wherein:
      L₁ is at one of the 3′-position and 4′-position of the diaryl ring D;
      R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or
      R₇ and R₈ together are: 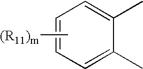
         wherein m is an integer from 1 to 3, and R₁₁ is selected from one of H and —CONHR₁₂NR₁₃R₁₄, wherein:
         R₁₂ is alkyl, and R₁₃ and R₁₄ are each independently selected from one of H and alkyl; or
            a pharmaceutically acceptable salt thereof.

In some embodiments, the method comprises a compound of Formula (I) selected from the group consisting of: 2-(4′-carbamimidoyl-biphenyl-3-yl)-1H-benzimidazole-5-carboxamidine (23); 2-[3-(5-carbamimidoyl-pyridin-2-yl)-phenyl]-1H-benzimidazole-5-carboxamidine (40); N-hydroxy-2-[4-hydroxy-4′-(N-hydroxycarbamimidoyl)-biphenyl-3-yl]-1H-benzimidazole-5-carboxamidine (3); 2-(4′-carbamimidoyl-4-hydroxy-biphenyl-3-yl)-1H-benzimidazole-5-carboxamidine (4); 2-(3′-carbamimidoyl-4-hydroxy-biphenyl-3-yl)-1H-benzimidazole-5-carboxamidine (8); 2-(4′-carbamimidoyl-4-hydroxy-5-methoxybiphenyl-3-yl)-1H-benzimidazole-5-carboxamidine (12); 2-(4′-carbamimidoyl-4-methoxy-biphenyl-3-yl)-1H-benzimidazole-5-carboxamidine; 2-(4′-carbamimidoyl-2′-methyl-biphenyl-3-yl)-1H-benzimidazole-5-carboxamidine (28); 2-[5-(5-carbamimidoyl-pyridin-2-yl)-2-methoxyphenyl]-1H-benzimidazole-5-carboxamidine (36); and 2-(4′-carbamimidoyl-2-hydroxy-biphenyl-3-yl)-1H-benzimidazole-5-carboxamidine (16).

Further, with regard to the presently described method embodiments, compounds of Formula (II) are defined as having a structure as follows: 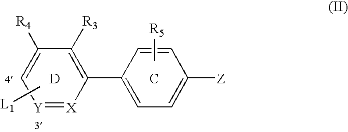
wherein:
   X and Y are each independently selected from the group consisting of CH, N, O and S, and Y can be present or absent;
   R₃, R₄, and R₅ are each independently selected from the group consisting of H, alkyl, halogen, hydroxyl, alkoxyl, aryloxyl, and aralkoxyl;
   Z is selected from one of: 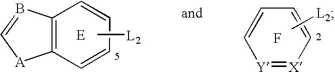
      wherein:
         A is selected from the group consisting of O, S, and NR₆, and wherein R₆ is selected from one of H and alkyl;
         B is selected from the group consisting of O, S, and N;
   X′ and Y′ are each independently selected from the group consisting of CH, N, O and S, and Y′ can be present or absent;
   L₁ and L₂ are each independently selected from the group consisting of: 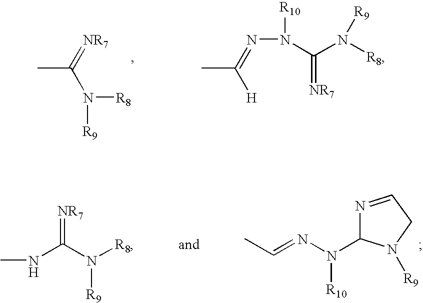
   wherein:
      L₁ is at one of the 3′-position and the 4′-position of the diaryl ring D;
      R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
      R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
      R₇ and R₈ together represent a C₂ to C₁₀ alkyl, hydroxyalkyl, or alkylene; or
      R₇ and R₈ together are: 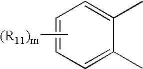
         wherein:
         m is an integer from 1 to 3, and R₁₁ is selected from one of H and —CONHR₁₂NR₁₃R₁₄, wherein R₁₂ is alkyl and R₁₃ and R₁₄ are each independently selected from one of H and alkyl; or
            a pharmaceutically acceptable salt thereof.

In some embodiments, the method comprises a compound of Formula (II) selected from the group consisting of: 2-(4′-carbamimidoyl-2′-methylbiphenyl-4-yl)-1H-benzimidazole-5-carboxamidine (52); 2-[4-(5-carbamimidoyl-pyridin-2-yl)-phenyl]-1H-benzimidazole-5-carboxamidine (44); 2-(4′-carbamimidoyl-3′-fluoro-biphenyl-4-yl)-1H-benzimidazole-5-carboxamidine (32); and 2-(4′-carbamimidoyl-2′-hydroxy-biphenyl-4-yl)-1H-benzimidazole-5-carboxamidine (48).

The subject treated in the presently disclosed subject matter in its many embodiments is desirably a human subject, although it is to be understood the methods described herein are effective with respect to all vertebrate species, which are intended to be included in the term “subject”. The methods described herein are particularly useful in the treatment and/or prevention of infectious diseases in warm-blooded vertebrates. Thus, the methods may be used as treatment for mammals and birds.

More particularly, provided is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, embodiments of the methods described herein include the treatment of livestock, including, but not limited to, domesticated swine (pigs and hogs), ruminants, horses, poultry, and the like.

### EXAMPLES

The following Examples have been included to illustrate modes of the presently disclosed subject matter. Certain aspects of the following Examples are described in terms of techniques and procedures found or contemplated to work well in the practice of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Methods and Materials For Examples 1-13

Melting points were recorded using a Thomas-Hoover (Uni-Melt®) (Thomas Scientific, Swedesboro, N.J., United States of America) capillary melting point apparatus and are uncorrected. TLC analysis was carried out on silica gel 60 F₂₅₄ precoated aluminum sheets and detected under UV light. ¹H and ¹³C NMR spectra were recorded employing a Varian GX400 or Varian Unity Plus 300 spectrometer (Varian, Inc., Palo Alto, Calif., United States of America), and chemical shifts (δ) are in ppm relative to TMS as internal standard. Mass spectra were recorded on a VG Analytical 70-SE spectrometer (VG Analytical, Ltd., Manchester, United Kingdom) for pure components. Elemental analyses were obtained from Atlantic Microlab Inc. (Norcross, Ga., United States of America) and are within ±0.4 of the theoretical values. All chemicals and solvents were purchased from Aldrich Chemical Co. (Milwaukee, Wis., United States of America) or Fisher Scientific (Fairlawn, N.J., United States of America) or Frontier Scientific (Logan, Utah, United States of America) or Lancaster Synthesis, Inc. (Windham, N.H., United States of America).

### Example 1

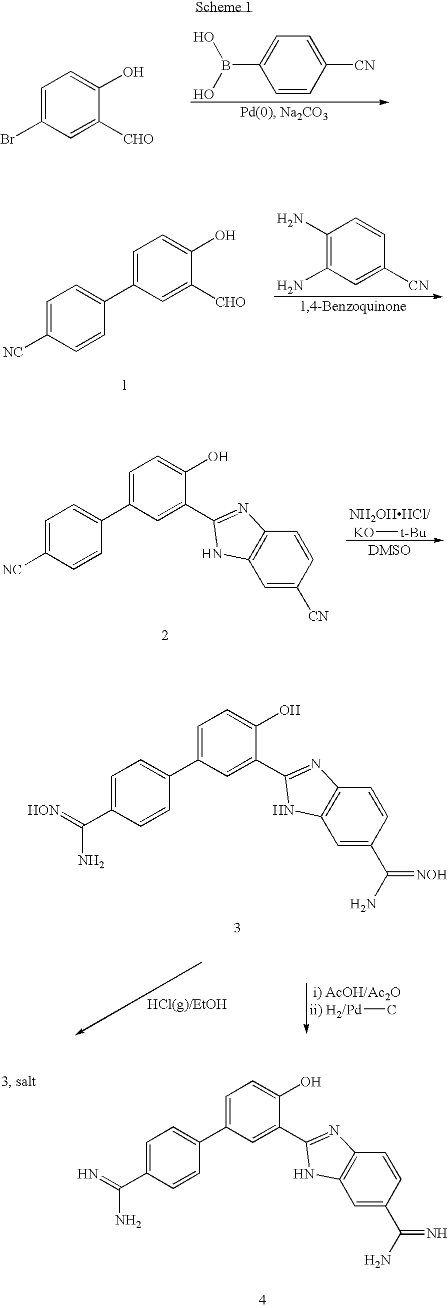

3′-formyl-4′-hydroxy-biphenyl-4-carbonitrile (1). Referring now to Scheme 1, 4 mL of a 2 M aqueous solution of Na₂CO₃ was added to a stirred solution of 5-bromo-2-hydroxy-benzaldehyde (804 mg, 4 mmol) and tetrakis(triphenylphosphine) palladium (230 mg) in toluene (8 mL) under a nitrogen atmosphere, followed by 4-cyanophenyl boronic acid (657 mg, 4.8 mmol) in 4 mL of methanol. The vigorously stirred mixture was warmed to 80° C. for 12 h. The solvent was evaporated, the precipitate was partitioned between methylene chloride (150 mL) and 2 M aqueous Na₂CO₃ (12 mL) containing 2 mL of concentrated ammonia. The organic layer was dried (Na₂SO₄), and then concentrated to dryness under reduced pressure to afford 1 in 62% yield; mp 143.5-144° C. (EtOH). ¹H NMR (DMSO-d₆); δ 7.13 (d, J=8.7 Hz, 1H), 7.83-7.91 (m, 4H), 7.94 (d, J=8.7 Hz, 1H), 8.02 (s, 1H), 10.30 (s, 1H) 11.00 (brs, 1H). ¹³C NMR; δ 191.0, 161.1, 143.3, 134.7, 132.8, 129.3, 127.3, 126.8, 122.6, 118.8, 118.2, 109.5. MS (m/z, rel. int.); 223 (M⁺, 100), 204 (10), 177 (15), 164 (10), 140 (15). High resolution calcd. for C₁₄H₉NO₂ ms 223.06333. Observed 223.06219. Anal. (C₁₄H₉NO₂) C, H, N.

2-(4′-cyano-4-hydroxy-biphenyl-3-yl)-1H-benzimidazole-5-carbonitrile (2). A solution of 1 (557.5 mg, 2.5 mmol), 3,4-diaminobenzonitrile (332.5 mg, 2.5 mmol), and benzoquinone (270.2 mg, 2.5 mmol) in ethanol (40 mL) was allowed to reflux under nitrogen for overnight. The reaction mixture was distilled off under reduced pressure. The residue was triturated with ether and filtered off to afford 2 in 90%, mp>340° C. ¹H NMR (DMSO-d₆); δ 7.20 (d, J=8.4 Hz, 1H), 7.68 (d, J=8.4 Hz, 1H), 7.86-7.98 (m, 6H), 8.28 (s, 1H), 8.57 (s, 1H), 12.80 (brs, 1H), 13.65 (brs, 1H). ¹³C NMR; δ 158.4, 153.9, 143.5, 132.9, 131.0, 129.3, 126.7, 125.7, 119.7, 118.9, 118.1, 112.8, 109.4, 104.5. MS(m/z, rel. int.); 336 (M⁺, 100), 307 (25), 280 (5), 164 (10). High resolution calcd. for C₂₁H₁₂N₄O ms 336.10111. Observed 336.10189. Anal. (C₂₁H₁₂N₄O-0.25H₂O) C, H, N.

N-hydroxy-2-[4-hydroxy-4′-(N-hydroxycarbamimidoyl)-biphenyl-3-yl]-1H-benzimidazole-5-carboxamidine (3). A mixture of hydroxylamine hydrochloride (1.04 g, 15 mmol. 10 eq.) in anhydrous DMSO (8 mL) was cooled to 5° C. under nitrogen, and potassium t-butoxide (1.68 g, 15 mmol, 10 eq.) was added in portions. The mixture was stirred for 30 min. This mixture was added to the bis cyanoderivative 2 (1.5 mmol, 1 eq.). The reaction mixture was stirred overnight at room temperature. The reaction mixture was then poured slowly onto ice water (50 mL water and 50 mL ice). The precipitate was filtered and washed with water to afford 3 (free base) in 94% yield; mp 319-322° C. ¹H NMR (DMSO-d₆); δ 5.87 (s, 4H), 7.13 (d, J=8.4 Hz, 1H), 7.63 (d, J=8.4 Hz, 1H), 7.69-8.02 (m, 7H), 8.45 (s, 1H), 9.60 (s, 1H), 9.63 (s, 1H), 13.20 (brs, 1H), 13.41 (brs, 1H).

3, salt. Mp 301-303° C.^{dec}. Anal. (C₂₁H₁₈N₆O₃-3.0HCl-2.8H₂O)C, H, N.

2-(4′-carbamimidoyl-4-hydroxy-biphenyl-3-yl)-1H-benzimidazole-5-carboxamidine acetate salt (4). To a solution of 3 (402 mg, 1 mmol) in glacial acetic acid (10 mL) was slowly added acetic anhydride (0.35 mL). After stirring for overnight (TLC indicated complete acylation of the starting material), 10% palladium on carbon (80 mg) was then added. The mixture was placed on Parr hydrogenation apparatus at 50 psi for 4 h at room temperature. The mixture was filtered through Hyflo and the filter pad washed with water. The filtrate was evaporated under reduced pressure and the precipitate was collected and washed with ether to give 4 in 78.5% yield, mp 223-224° C.^{dec}. ¹H NMR (D₂O/DMSO-d₆); δ 1.80 (s, 3xCH₃), 7.00 (d, J=8.4 Hz, 1H), 7.48 (d, J=8.4 Hz, 1H), 7.64-7.67 (m, 2H), 7.90 (s, 4H), 8.08 (s, 1H), 8.62 (s, 1H). Anal. (C₂₁H₁₈N₆O-3.0CH₃CO₂H-1.8H₂O)C, H, N.

### Example 2

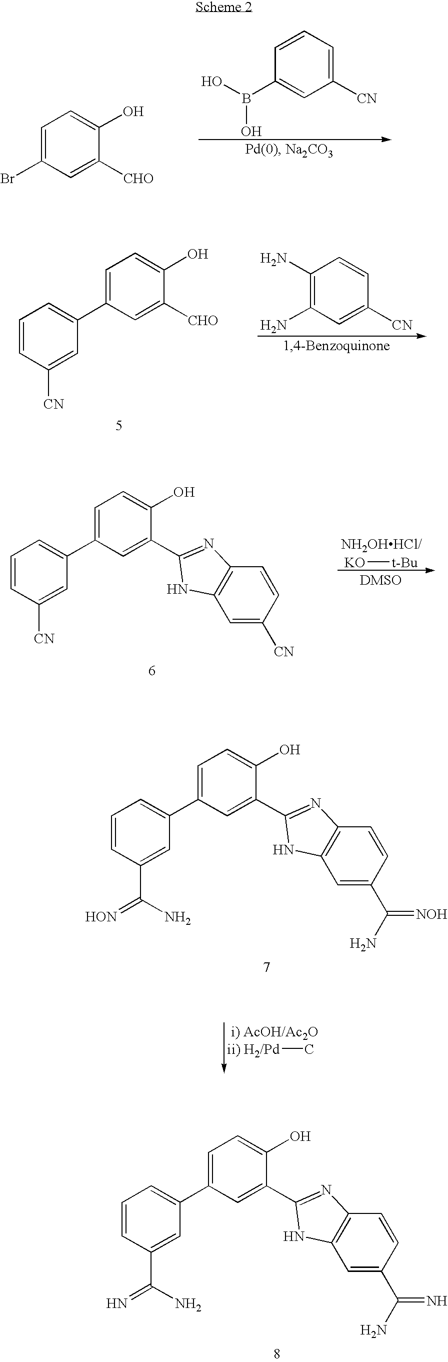

3′-formyl-4′-hydroxy-biphenyl-3-carbonitrile (5). Referring now to Scheme 2, the same procedure described for compound 1 was used employing 3-cyanophenyl boronic acid instead of 4-cyanophenyl boronic acid. Yield 70%; mp 139-140° C. (EtOH). ¹H NMR (DMSO-d₆); δ 7.12 (d, J=8.7 Hz, 1H), 7.62-7.81 (m, 2H), 7.92-8.02 (m, 3H), 8.14 (s, 1H), 10.32 (s, 1H) 11.00 (brs, 1H). ¹³C NMR; δ 191.3, 160.7, 140.0, 134.6, 130.8, 130.5, 130.1, 129.5, 129.2, 127.4, 122.5, 118.7, 118.0, 112.0. MS (m/z, rel. int.); 223 (M⁺, 100), 204 (10), 193 (5), 177 (20), 166 (15), 140 (15).

2-(3′-cyano-4-hydroxy-biphenyl-3-yl)-1H-benzimidazole-5-carbonitrile (6). The same procedure described for compound 2 was used, starting with compound 5. Yield 89%, mp 348-350° C. (EtOH). ¹H NMR (DMSO-d₆); δ 7.20 (d, J=8.4 Hz, 1H), 7.67-7.75 (m, 2H), 7.82-7.88 (m, 3H), 8.10 (d, J=8.4 Hz, 1H), 8.21 (s, 1H), 8.26 (s, 1H), 8.55 (s, 1H), 13.20 (brs, 2H). ¹³C NMR; δ 158.2, 154.0, 140.2, 130.8, 130.7, 130.5, 130.2, 129.4, 129.1, 126.3, 125.4, 119.7, 118.8, 118.0, 112.7, 112.1, 104.7. MS (m/z, rel. int.); 336 (M⁺, 100), 307 (20), 306 (12), 168 (5), 140 (5). High resolution mass calcd. for C₂₁H₁₂N₄O: 336.10111. Observed 336.10247. Anal. (C₂₁H₁₂N₄O-0.3H₂O)C, H.

N-hydroxy-2-[4-hydroxy-3′-(N-hydroxycarbamimidoyl)-biphenyl-3-yl]-1H-benzimidazole-5-carboxamidine (7). The same procedure described for compound 3 was used, starting with compound 6. Yield 97%, mp 352-355° C. ¹H NMR (DMSO-d₆); δ 5.89 (s, 4H), 7.20 (d, J=8.4 Hz, 1H), 7.58 (d, J=8.4 Hz, 1H), 7.63-8.90 (m, 6H), 8.09 (s, 1H), 8.50 (s, 1H), 9.70 (s, 2H), 13.20 (brs, 1H), 13.44 (brs, 1H). ¹³C NMR; δ 157.7, 152.4, 151.2, 150.9, 139.3, 134.1, 131.2, 130.2, 128.7, 126.7, 124.5, 124.1, 123.4, 117.8, 115.0, 112.8, 108.6.

2-(3′-carbamimidoyl-4-hydroxy-biphenyl-3-yl)-1H-benzimidazole-5-carboxamidine acetate salt (8). The same procedure described for compound 4 was used, starting with compound 7. Yield 80%. mp 208-209° C.^{dec}. ¹H NMR (D₂O/DMSO-d₆); δ 1.80 (s, 3xCH₃), 7.02 (d, J=8.4 Hz, 1H), 7.47 (d, J=8.4 Hz, 1H), 7.62-7.72 (m, 4H), 8.00 (d, J=7.2 Hz, 1H), 8.07 (s, 1H), 8.11 (s, 1H), 8.66 (s, 1H). Anal. (C₂₁H₁₈N₆O-3.0CH₃CO₂H-1.0H₂O)C, H, N.

### Example 3

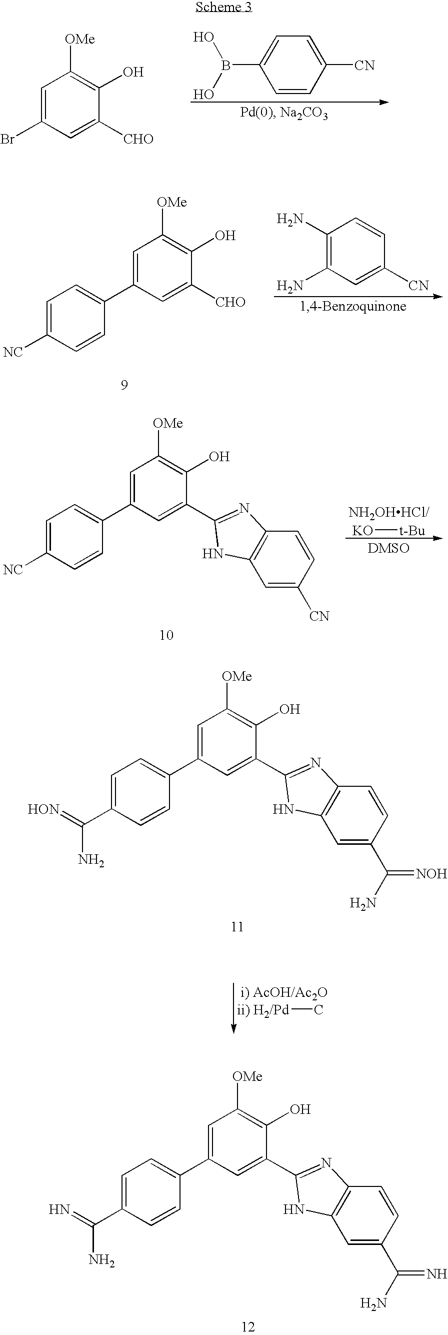

5′-formyl-4′-hydroxy-3′-methoxy-biphenyl-4-carbonitrile (9). Referring now to Scheme 3, the same procedure described for compound 1 was used, employing 5-bromo-2-hydroxy-3-methoxybenzaldehyde instead of 5-bromo-2-hydroxybenzaldehyde. Yield 57%; mp 177-177.5° C. ¹H NMR (DMSO-d₆); δ 3.97 (s, 3H), 7.61 (s, 2H), 7.90 (s, 4H), 10.33 (s, 1H), 10.57 (brs, 1H). ¹³C NMR; δ 191.3, 151.2, 149.0. 143.7, 132.7, 129.2, 127.1, 122.6, 118.9, 118.3, 115.7, 109.5, 56.3. MS (m/z, rel. int.); 253 (M⁺, 100), 210 (8), 207 (10), 177 (10), 154 (15), 127 (18). High resolution mass calcd. for C₁₅H₁₁NO₃: 253.07389. Observed 253.07181. Anal. (C₁₅H₁₁NO₃) C, H.

2-(4′-cyano-4-hydroxy-5-methoxy-biphenyl-3-yl)-1H-benzimidazole-5-carbonitrile (10). The same procedure described for compound 2 was used, starting with compound 9. Yield 79%, mp 334-335° C. ¹H NMR (DMSO-d₆); δ 3.98 (s, 3H), 7.49 (s, 1H), 7.68 (d, J=8.4 Hz, 1H), 7.84 (d, J=8.4 Hz, 1H), 7.94-8.00 (m, 4H), 8.13 (s, 1H), 8.25 (s, 1H), 13.2 (brs, 2H). ¹³C NMR; δ 154.2, 149.1, 148.9, 143.8, 132.7, 129.0, 126.9, 126.8, 119.6, 118.9, 116.8, 112.8, 112.3, 109.4, 104.7, 99.4, 56.0. MS (m/z, rel. int.); 366 (M⁺, 100), 348 (42), 337 (20), 323 (30), 307 (10). High resolution mass calcd. for C₂₂H₁₄N₄O₂: 366.11168. Observed 366.11188.

N-hydroxy-2-[4-hydroxy-4′-(N-hydroxycarbamimidoyl)-5-methoxy-biphenyl-3-yl]-1H-benzimidazole-5-carboxamidine (11). The same procedure described for compound 3 was used, starting with compound 10. Yield 99%, mp 319-321° C. ¹H NMR (DMSO-d₆); δ 4.00 (s, 3H), 5.89 (s, 4H), 7.43 (s, 1H), 7.59-7.74 (m, 2H), 7.82-7.88 (m, 4H), 8.05 (s, 1H), 8.09 (s, 1H), 9.61 (s, 1H), 9.69 (s, 1H), 13.40 (brs, 2H).

2-(4′-carbamimidoyl-4-hydroxy-5-methoxy-biphenyl-3-yl)-1H-benzimidazole-5-carboxamidine acetate salt (12). The same procedure described for compound 4 was used, starting with compound 11. Yield 75%, mp 230-231° C.^{dec}. ¹H NMR (D₂O/DMSO-d₆); δ 1.80 (s, 3xCH₃), 3.93 (s, 3H), 7.27 (s, 1H), 7.47 (d, J=8.4 Hz, 1H), 7.63 (d, J=8.4 Hz, 1H), 7.87-7.97 (m, 4H), 8.07 (s, 1H), 8.24 (s, 1H). MS (m/z, rel. int., EI/isobutane), 371 (M⁺-NH3, 10), 366 (50), 351 (10), 336 (100). Anal. (C₂₂H₂₀N₆O₂.3.0CH₃CO₂H-2.1H₂O)C, H, N.

### Example 4

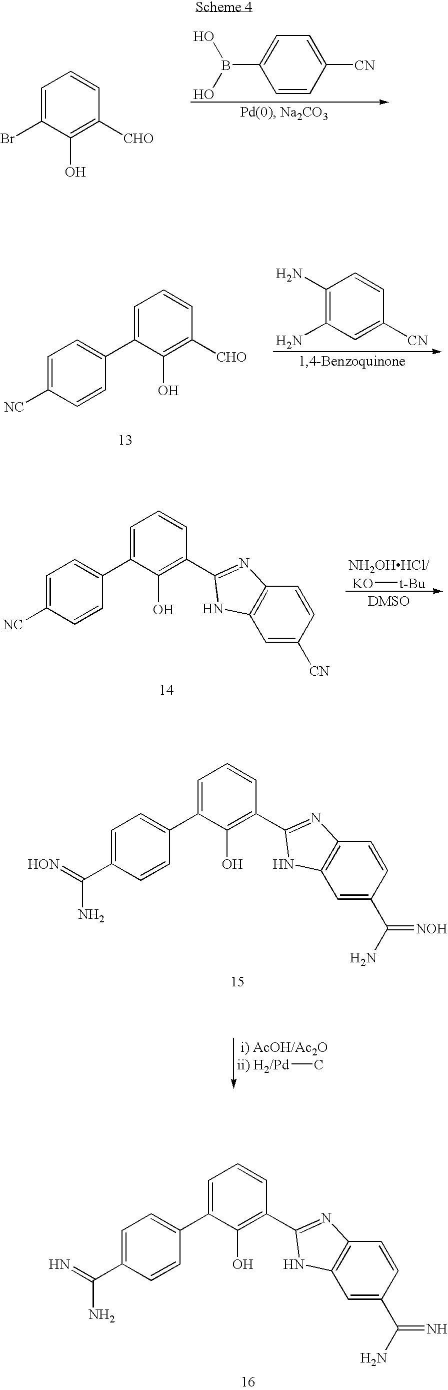

3′-formyl-2′-hydroxy-biphenyl-4-carbon itrile (13). Referring now to Scheme 4, the same procedure described for compound I was used, employing 3-bromo-2-hydroxy-benzaldehyde instead of 5-bromo-2-hydroxy-benzaldehyde. Yield 58%; mp 120-121° C. (hexanes/ether). ¹H NMR (CDCl₃); δ 7.25 (t, J=7.8 Hz, 1H), 7.60-7.70 (m, 2H), 7.73-7.80 (m, 4H), 9.97 (s, 1H), 11.64 (s, 1H). MS (m/z, rel. int.); 223 (M⁺, 100), 204 (20), 195 (25), 177 (25), 140 (20). High resolution mass calcd. for C₁₄H₉NO₂: 223.06333. Observed 223.06256.

2-(4′-cyano-2-hydroxy-biphenyl-3-yl)-1H-benzimidazole-5-carbonitrile (14). The same procedure described for compound 2 was used, starting with compound 13. Yield 84%, mp 318-320° C. ¹H NMR (DMSO-d₆); δ 7.18 (t, J=7.5 Hz, 1H), 7.56 (d, J=7.5 Hz, 1H), 7.71 (d, J=8.4 Hz, 1H), 7.78 (d, J=7.5 Hz, 1H), 7.84 (d, J=8.1 Hz, 2H), 7.91 (d, J=8.1 Hz, 2H), 8.14 (d, J=8.4 Hz, 1H), 8.28 (s, 1H), 13.70 (brs, 1H), 13.90 (brs, 1H). MS (m/z, rel. int.); 337 (M⁺+1, 68), 309 (100), 293 (40). Anal. (C₂₁H₁₂N₄O)C, H, N.

N-hydroxy-2-[2-hydroxy-4′-(N-hydroxycarbamimidoyl)-biphenyl-3-yl]-1H-benzimidazole-5-carboxamidine (15). The same procedure described for compound 3 was used, starting with compound 14. Yield 100%, mp 322-325° C.^{dec}. ¹H NMR (DMSO-d₆); δ 6.00 (s, 4H), 7.13 (t, J=7.8 Hz, 1H), 7.49 (d, J=7.2 Hz, 1H), 7.61-7.77 (m, 6H), 7.88-8.08 (m, 2H), 9.70 (s, 2H), 13.40 (br s, 1H), 13.90 (br s, 1H).

2-(4′-carbamimidoyl-2-hydroxy-biphenyl-3-yl)-1H-benzimidazole-5-carboxamidine acetate salt (16). The same procedure described for compound 4 was used, starting with compound 15. Yield 90%, mp 228-229.5° C.^{dec}. ¹H NMR (D₂O/DMSO-d₆); δ 1.80 (s, 2.8xCH₃), 6.97 (t, J=7.8 Hz, 1H), 7.35 (d, J=7.5 Hz, 1H), 7.45 (d, J=7.5 Hz, 1H), 7.60 (d, J=7.8 Hz, 1H), 7.86 (d, J=8.4 Hz, 2H), 7.93 (d, J=8.4 Hz, 2H), 8.05 (s, 1H), 8.27 (d, J=7.8 Hz, 1H). MS (m/z, rel. int., EI/isobutane); 371 (M⁺+1, 5), 354 (5), 337(100), 307 (10). Anal. (C₂₁H₁₈N₆O-2.8CH₃CO₂H-0.8H₂O-0.5C₂H₅OH)C, H, N.

### Example 5

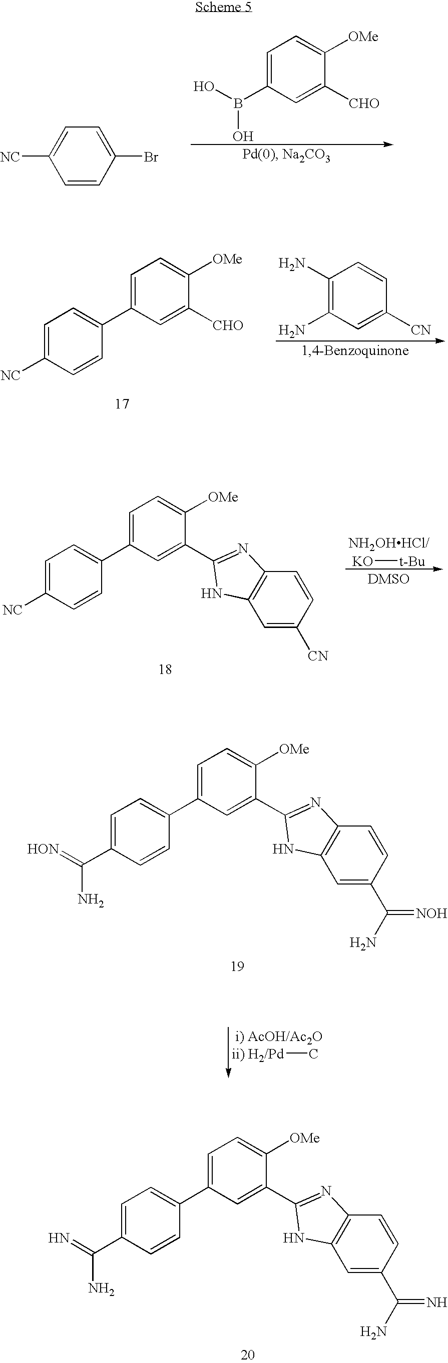

3′-formyl-4′-methoxy-biphenyl-4-carbonitrile (17). Referring now to Scheme 5, to a stirred solution of 4-bromobenzonitrile (5 mmol), and tetrakis(triphenylphosphine) palladium (288 mg) in toluene (10 mL) under a nitrogen atmosphere was added 5 mL of a 2 M aqueous solution of Na₂CO₃ followed by 3-formyl-4-methoxy-phenyl boronic acid (1080 mg, 6 mmol) in 5 mL of methanol. The vigorously stirred mixture was warmed to 80° C. for 12 h. The solvent was evaporated, the precipitate was partitioned between methylene chloride (200 mL) and 2 M aqueous Na₂CO₃ (15 mL) containing 3 mL of concentrated ammonia. The organic layer was dried (Na₂SO₄), and then concentrated to dryness under reduced pressure to afford 17 in 70% yield; mp 146-147° C. (SiO₂, hexanes/EtOAc, 90:10). ¹H NMR (DMSO-d₆); δ 4.00 (s, 3H), 7.39 (d, J=9.0 Hz, 1H), 7.88-7.94 (m, 4H), 8.03 (d, J=2.7 Hz, 1H), 8.09 (dd, J=9.0, 2.7 Hz, 1H), 10.40 (s, 1H). ¹³C NMR; δ 188.9, 161.7, 143.1, 134.7, 132.8, 130.5, 127.0, 126.2, 124.4, 118.8, 113.7, 109.8, 56.2. MS (m/z, rel.int.); 237 (M⁺, 100), 220 (20), 208 (10), 191 (25), 177 (35), 140 (20).

2-(4′-cyano-4-methoxy-biphenyl-3-yl)-1H-benzimidazole-5-carbonitrile (18). The same procedure described for compound 2 was used, starting with compound 17. Yield 88%, mp 289-290° C. ¹H NMR (DMSO-d₆); δ 4.09 (s, 3H), 7.43 (d, J=8.4 Hz, 1H), 7.61 (d, J=8.4 Hz, 1H), 7.81 (s, 1H), 7.94-8.22 (m, 6H), 8.67 (s, 1H), 12.63 (s, 1H). ¹³C NMR; δ 157.5, 151.1, 143.5, 142.1, 137.9, 132.9, 130.9, 130.7, 128.3, 127.1, 125.7, 125.6, 123.4, 120.1, 118.9, 117.7, 113.3, 113.2, 109.6, 103.8, 56.3. MS (m/z, rel.int.); 350 (M⁺, 100), 321 (40), 306 (12), 144 (50). High resolution mass calcd. for C₂₂H₁₄N₄O: 350.11676. Observed 350.11569.

N-hydroxy-2-[4′-(N-hydroxycarbamimidoyl)-4-methoxy-biphenyl-3-yl]-1H-benzimidazole-5-carboxamidine (19). The same procedure described for compound 3 was used, starting with compound 18. Yield 95%, mp>340° C. ¹H NMR (DMSO-d₆); δ 4.09 (s, 3H), 6.09 (s, 2H), 6.71 (s, 2H), 7.38 (d, J=8.4 Hz, 1H), 7.58 (d, J=8.4 Hz, 1H), 7.69 (d, J=8.4 Hz, 1H), 7.75 (d, J=8.7 Hz, 2H), 7.81 (d, J=8.7 Hz, 2H), 7.87 (dd, J=8.4, 2.4 Hz, 1H), 7.99 (s, 1H), 8.64 (d, J=2.4 Hz, 1H), 9.80 (5, 1H), 10.0 (s, 1H), 12.55 (brs, 1H). ¹³C NMR; δ 156.6, 151.0, 150.0, 139.7, 137.9, 132.1, 131.5, 129.6, 127.6, 126.1, 125.9, 120.4, 118.0, 112.9, 56.1. FABMS (m/z, rel.int.); 417 (M⁺+1, 100), 401 (58), 394 (30), 368 (20), 350 (10). HRMS calcd. for C₂₂H₂₁N₆O₃: 417.16751. Observed 417.16760.

2-(4′-carbamimidoyl-4-methoxy-biphenyl-3-yl)-1H-benzimidazole-5-carboxamidine acetate salt (20). The same procedure described for compound 4 was used, starting with compound 19. Yield 62%, mp 220-221° C. ¹H NMR (D₂O/DMSO-d₆); δ 1.78 (s, 2.6xCH₃), 4.11 (s, 3H), 7.43 (d, J=8.4 Hz, 1H), 7.65 (d, J=8.4 Hz, 1H), 7.81 (d, J=8.4 Hz, 1H), 7.87-7.96 (m, 4H), 8.15 (dd, J=8.4, 2.4 Hz, 1H), 8.31 (s, 1H), 8.69 (d, J=2.4 Hz, 1H). Anal. (C₂₂H₂₀N₆O-2.6CH₃CO₂H-2.0H₂O)C, H, N.

### Example 6

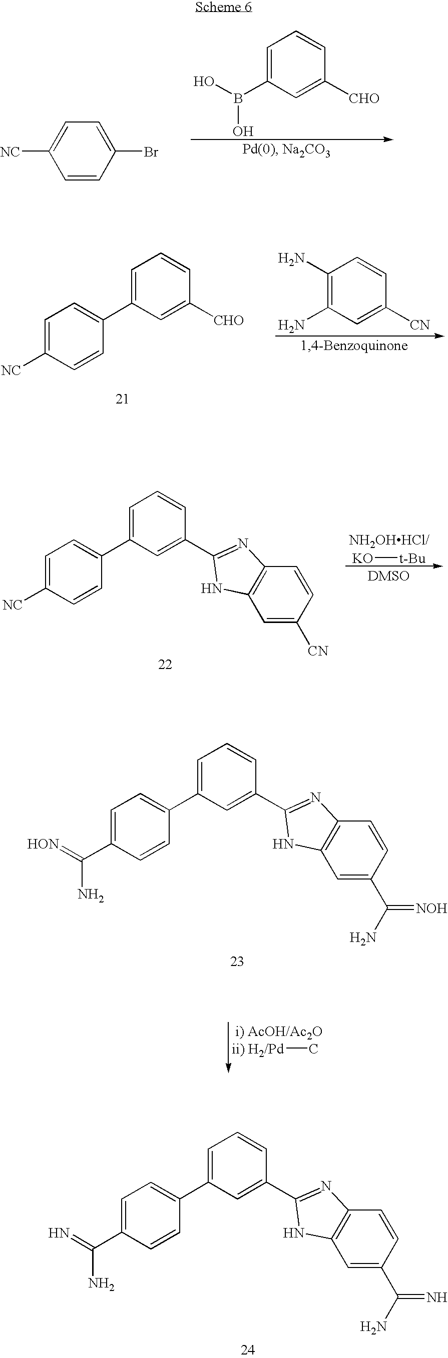

3′-formylbiphenyl-4-carbonitrile (21). Referring now to Scheme 6, the same procedure described for compound 17 was used, employing 3-formylphenyl boronic acid instead of 3-formyl-4-methoxy-phenyl boronic acid. Yield 80%, mp 122-123° C. ¹H NMR (DMSO-d₆); δ 7.72-7.78 (m, 1H), 7.98-8.03 (m, 5H), 8.11 (m, 1H), 8.29 (s, 1H), 10.13 (s, 1H). ¹³C NMR; δ 193.0, 143.3, 139.0, 136.9, 132.98, 132.95, 130.0, 129.0, 128.5, 127.7, 118.7, 110.6. Anal. (C₁₄H₉NO)C, H.

2-(4′-cyanobiphenyl-3-yl)-1H-benzimidazole-5-carbonitrile (22). The same procedure described for compound 2 was used, starting with compound 21. Yield 81%, mp 303-304° C. ¹H NMR (DMSO-d₆); δ 7.63 (d, J=8.4 Hz, 1H), 7.73 (t, J=7.8 Hz, 1H), 7.80 (d, J=8.4 Hz, 1H), 7.96 (d, J=7.8 Hz, 1H), 8.00-8.06 (m, 4H), 8.19 (s, 1H), 8.30 (d, J=7.8 Hz, 1H), 8.57 (s, 1H), 13.60 (brs, 1H). ¹³C NMR; δ 153.9, 143.7, 139.0, 132.9, 130.07, 130.01, 129.2, 127.7, 127.1, 125.8, 125.4, 119.9, 118.7, 110.5, 104.1. MS (m/z, rel.int.); 320 (M⁺, 100), 291 (5), 204 (5), 177 (8), 151 (3). High resolution mass calcd. for C₂₁H₁₂N₄: 320.10620. Observed 320.10644.

N-hydroxy-2-[4′-(N-hydroxycarbamimidoyl)-biphenyl-3-yl]-1H-benzimidazole-5-carboxamidine (23). The same procedure described for compound 3 was used, starting with compound 22. Yield 93%, mp 317-319° C. ¹H NMR (DMSO-d₆); δ 5.89 (s, 2H), 5.92 (s, 2H), 7.60-7.70 (m, 3H), 7.80-7.98 (m, 5H), 8.00 (s, 1H), 8.21 (d, J=7.8 Hz, 1H), 8.52 (s, 1H), 9.60 (s, 1H), 9.74 (s, 1H), 13.11 (brs, 1H). Anal. (C₂, H₁₈N₆O₂.1.0H₂O)C, H.

2-(4′-carbamimidoyl-biphenyl-3-yl)-1H-benzimidazole-5-carboxamidine acetate salt (24). The same procedure described for compound 4 was used, starting with compound 23. Yield 72%, mp 211-212° C. ¹H NMR (D₂O/DMSO-d₆); δ 1.78 (s, 3xCH₃), 7.60 (d, J=8.4 Hz, 1H), 7.69 (t, J=7.8 Hz, 1H), 7.76 (d, J=8.4 Hz, 1H), 7.91 (d, J=7.8 Hz, 1H), 7.96 (d, J=8.7 Hz, 2H), 8.03 (d, J=8.7 Hz, 2H), 8.12 (s, 1H), 8.33 (d, J=7.8 Hz, 1H). 8.65 (s, 1H). Anal. (C₂₁H₁₈N₆.3.0CH₃CO₂H-2.0H₂O)C, H, N.

### Example 7

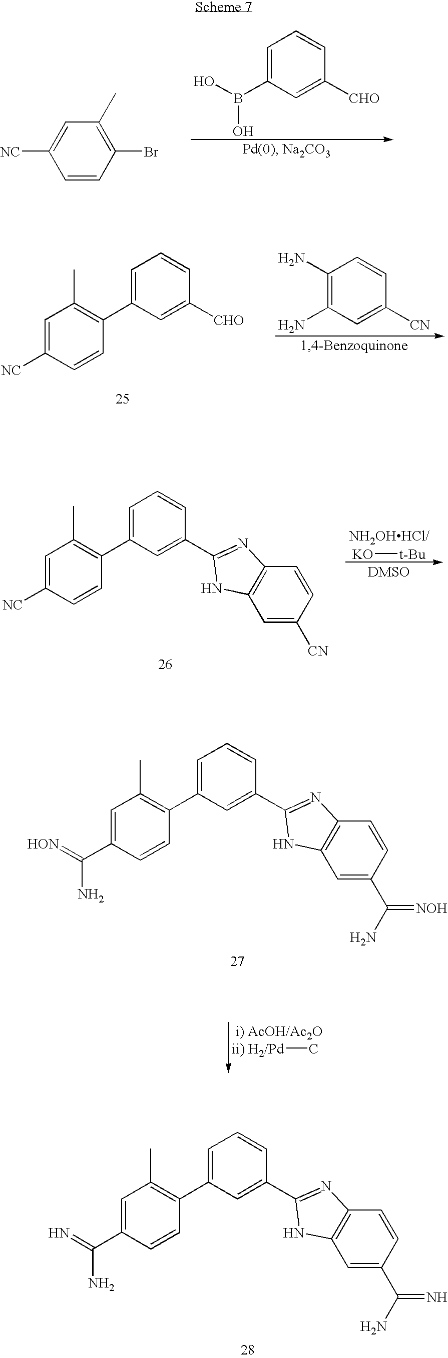

3′-formyl-2-methyl-biphenyl-4-carbonitrile. (25). Referring now to Scheme 7, the same procedure described for compound 21 was used, employing 4-bromo-3-methyl-benzonitrile instead of 4-bromobenzonitrile. Yield 82%, mp 86-86.5° C. ¹H NMR (DMSO-d₆); δ 2.27 (s, 3H), 7.47 (d, J=7.8 Hz, 1H), 7.71-7.79 (m, 3H), 7.85 (s, 1H), 7.90 (s, 1H), 7.95-7.98 (m, 1H), 10.08 (s, 1H). ³C NMR; δ 193.0, 144.8, 140.3, 136.8, 136.3, 134.7, 133.9, 130.5, 130.0, 129.8, 129.4, 128.4, 118.7, 110.6, 19.7. Anal. (C₁₅H₁₁NO)C, H.

2-(4′-cyano-2′-methyl-biphenyl-3-yl)-1H-benzimidazole-5-carbonitrile (26). The same procedure described for compound 2 was used, starting with compound 25. Yield 75%, mp 247-250° C. ¹H NMR (DMSO-d₆); δ 2.26 (s, 3H), 7.50-7.63 (m, 3H), 7.67-7.80 (m, 3H), 7.87 (s, 1H), 8.17 (d, J=7.8 Hz, 2H), 8.28 (d, J=7.8 Hz, 1H), 13.40 (brs, 1H). MS (m/z, rel.int.); 334 (M⁺, 100), 215 (5), 190 (20), 167 (6). High resolution mass calcd. for C₂₂H₁₄N₄: 334.12185. Observed 334.12142. Anal. (C₂₂H₁₄N₄.0.5H₂O)C, H.

N-hydroxy-2-[4′-(N-hydroxycarbamimidoyl)-2′-methyl-biphenyl-3-yl]-1H-benzimidazole-5-carboxamidine (27). The same procedure described for compound 3 was used, starting with compound 26. Yield 99%, mp 295-297° C.^{dec 1}H NMR (DMSO-d₆); δ 2.30 (s, 3H), 5.87 (s, 4H), 7.32 (d, J=8.1 Hz, 1H), 7.50 (d, J=7.8 Hz, 1H), 7.61-7.97 (m, 6H), 8.15 (s, 1H), 8.21 (d, J=7.8 Hz, 1H), 9.59 (s, 1H), 9.68 (s, 1H), 13.02 (brs, 1H). ¹³C NMR; δ 151.5, 150.5, 141.4, 141.0, 134.5, 132.5, 130.3, 130.0, 129.3, 128.9, 127.3, 127.2, 126.7, 125.2, 123.0, 119.8, 118.2, 115.9, 110.7, 108.4, 20.2. FABMS (m/z, rel.int.); 401 (M⁺+1, 100), 386 (55), 368 (30), 352 (15), 335 (10). HRMS calcd. for C₂₂H₂₁N₆O₂: 401.17260. Observed 401.17287.

2-(4′-carbamimidoyl-2′-methyl-biphenyl-3-yl)-1H-benzimidazole-5-carboxamidine acetate salt (28). The same procedure described for compound 4 was used, starting with compound 27. Yield 83%, mp 201-203° C. ¹H NMR (D₂O/DMSO-d₆); δ 1.80 (s, 2.8xCH₃), 2.33 (s, 3H), 7.50-7.59 (m, 2H), 7.62-7.78 (m, 3H), 7.82-7.95 (m, 2H), 8.12 (8, 1H), 8.21-8.34 (m, 2H). Anal. (C₂₂H₂₀N₆.2.8CH₃CO₂H-2.3H₂O)C, H, N.

### Example 8

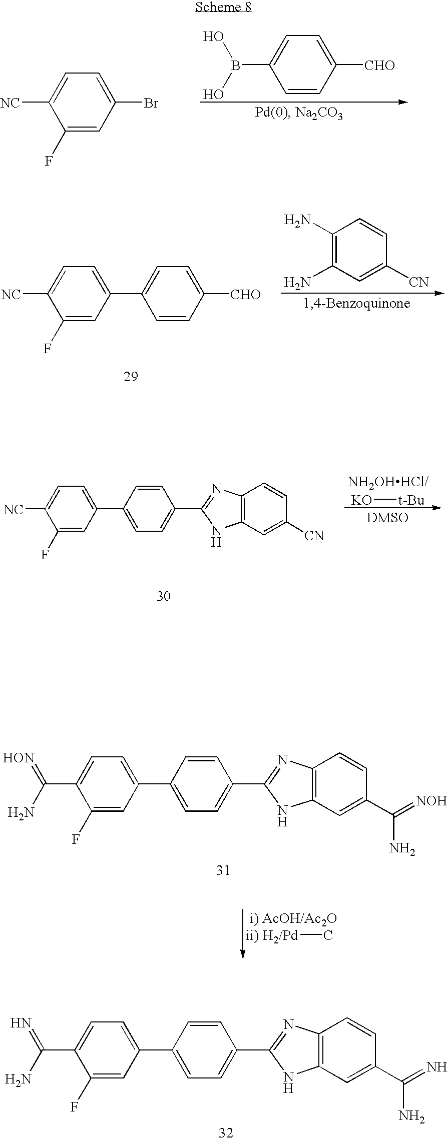

3-fluoro-4′-formylbiphenyl-4-carbonitrile (29). Referring now to Scheme 8, the same procedure described for compound 17 was used, employing 4-bromo-2-fluoro-benzonitrile instead of 4-bromobenzonitrile and 4-formylphenyl boronic acid instead of 3-formyl-4-methoxy-phenyl boronic acid.

Yield 78%, mp 186-187° C. ¹H NMR (DMSO-d₆); δ 7.83-7.87 (m, 1H), 7.99-8.09 (m, 6H), 10.09 (s, 1H). ¹³C NMR; δ 192.7, 164.5, 161.1, 146.2, 146.1, 142.4, 136.3, 134.4, 130.1, 128.0, 124.1, 115.1, 114.8, 113.9, 99.8, 99.6 (fluorine splitting). MS (m/z, rel.int.); 225 (M⁺, 75), 224 (100), 195 (25), 169 (20). Anal. (C₁₄H₈FNO)C, H.

2-(4′-cyano-3′-fluoro-biphenyl-4-yl)-1H-benzimidazole-5-carbonitrile (30). The same procedure described for compound 2 was used, starting with compound 29. Yield 84%, mp 302-305° C.^{dec}. ¹H NMR (DMSO-d₆); δ 7.62 (d, J=8.1 Hz, 1H), 7.78 (d, J=8.1 Hz, 1H), 7.88 (d, J=8.1 Hz, 1H), 8.00-8.07 (m, 4H), 8.17 (s, 1H), 8.33 (d, J=8.1 Hz, 2H), 13.60 (s, 1H). ¹³C NMR; δ 164.5, 161.2, 146.5, 146.4, 138.7, 134.3, 129.8, 127.9, 127.5, 123.5, 119.9, 115.5, 114.5, 114.3, 114.0, 104.1, 99.2, 99.0. MS (m/z, rel.int.); 338 (M⁺, 100), 222 (5), 195 (5), 169 (10). High resolution mass calcd. for C₂₁H₁₁N₄F: 338.09677. Observed 338.09778.

2-[3-fluoro-4′-(N-hydroxycarbamimidoyl)-biphenyl-4-yl]-N-hydroxy-1H-benzimidazole-5-carboxamidine (31). The same procedure described for compound 3 was used, starting with compound 30. Yield 96%, mp 281-283° C.^{dec}. ¹H NMR (DMSO-d₆); δ 5.86 (s, 4H), 7.60-7.74 (m, 6H), 7.98 (d, J=8.4 Hz, 2H), 8.32 (d, J=8.4 Hz, 2H), 9.60 (s, 1H), 9.74 (s, 1H), 13.09 (brs, 1H). ¹³C NMR; δ 161.7, 158.4, 151.7, 151.5, 148.0, 141.6, 141.5, 139.2, 130.4, 129.6, 127.2, 127.0, 122.2, 121.2, 121.0, 114.2, 113.9. MS (m/z, rel.int.); 405 (M⁺+1, 70), 203 (100).

2-(4′-carbamimidoyl-3′-fluoro-biphenyl-4-yl)-1H-benzimidazole-5-carboxamidine acetate salt (32). The same procedure described for compound 4 was used, starting with compound 31. Yield 85%, mp 210-212° C.^{dec}. ¹H NMR (D₂O/DMSO-d₆); δ 1.78 (s, 3xCH₃), 7.61 (d, J=8.4 Hz, 1H), 7.84-8.05 (m, 6H), 8.12 (s, 1H), 8.39 (d, J=8.4 Hz, 2H). Anal. (C₂₁H₁₇N₆F-3.0CH₃CO₂H-1.3H₂O)C, H, N.

### Example 9

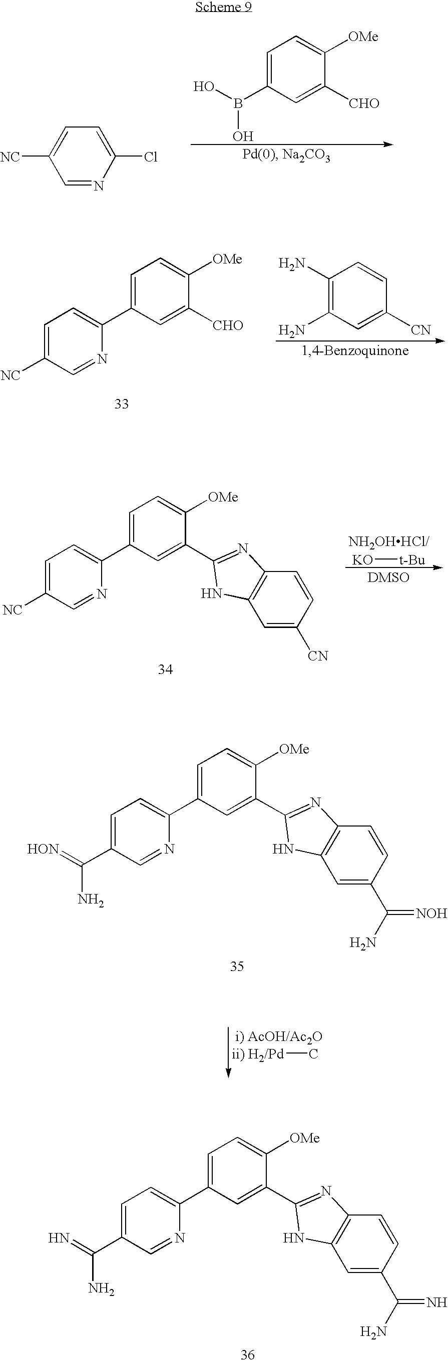

6-(3-formyl-4-methoxy-phenyl)-nicotinonitrile (33). Referring now to Scheme 9, the same procedure described for compound 17 was used, employing 6-chloronicotinonitrile instead of 4-bromobenzonitrile. Yield 66.5%; mp 197-198° C. (EtOH). ¹H NMR (CDCl₃); δ 4.00 (s, 3H), 7.16 (d, J=8.4 Hz, 1H), 7.87 (d, J=8.4 Hz, 1H), 8.00 (dd, J=8.4, 2.1 Hz, 1H), 8.41 (dd, J=8.4, 2.1 Hz, 1H), 8.46 (d, J=2.1 Hz, 1H), 8.92 (d, J=2.1 Hz, 1H), 10.57 (s, 1H). ¹³C NMR (DMSO-d₆); δ 188.5, 162.7, 157.5, 152.0, 140.4, 134.3, 129.1, 126.7, 124.3, 119.2, 116.8, 113.1, 106.7, 56.1. MS (m/z, rel.int.); 238 (M⁺, 100), 221 (32), 209 (15), 192 (25), 178 (25), 166 (20). High resolution mass calcd. for C₁₄H₁₀N₂O₂: 238.07423. Observed 238.07486.

2-[5-(5-cyanopyridin-2-yl)-2-methoxy-phenyl]-1H-benzimidazole-5-carbonitrile (34). The same procedure described for compound 2 was used, starting with compound 33. Yield 92%, mp 316.5-319° C. ¹H NMR (DMSO-d₆); δ 4.07 (s, 3H), 7.44 (d, J=8.4 Hz, 1H), 7.58-7.87 (m, 2H), 8.03-8.36 (m, 4H), 9.09 (s, 1H), 9.20 (d, J=2.1 Hz, 1H), 12.60 (brs, 1H). ¹³C NMR; δ 158.7, 157.9, 152.4, 151.4, 140.8, 130.8, 129.6, 129.1, 125.4, 119.9, 119.4, 117.5, 117.2, 112.9, 106.8, 103.8, 56.3. MS (m/z, rel.int.); 351 (M⁺, 100), 322 (40), 293 (10), 143 (35). HRMS calcd. for C₂₁H₁₃N₅O: 351.11201. Observed 351.11067. Anal. (C₂₁H₁₃N₅O)C, H, N.

N-hydroxy-2-{5-[5-(N-hydroxycarbamimidloyl)-pyridin-2-yl]-2-methoxy-phenyl}-1H-benzimidazole-5-carboxamidine (35). The same procedure described for compound 3 was used, starting with compound 34. Yield 99%, Mp 276-279° C.^{dec}. ¹H NMR (DMSO-d₆); δ 4.08 (s, 3H), 5.83 (s, 2H), 6.02 (s, 2H), 7.37 (d, J=8.4 Hz, 1H), 7.58-7.68 (m, 2H), 7.95 (s, 1H), 8.02 (d, J=8.4 Hz, 1H), 8.11 (d, J=8.4 Hz, 1H), 8.22 (dd, J=8.4, 2.1 Hz, 1H), 8.95 (s, 1H), 9.13 (d, J=2.1 Hz, 1H), 9.57 (s, 1H), 9.87 (s, 1H), 12.30 (brs, 1H). ¹³C NMR; δ 157.6, 155.2, 152.1, 151.9, 149.7, 148.8, 146.5, 133.9, 130.9, 129.4, 128.0, 127.2, 118.8, 118.2, 112.6, 56.1. FABMS (m/z, rel.int.); 418 (M⁺+1, 70), 401 (40), 385 (23), 327 (50), 237 (100). HRMS calcd. for C₂₁H₂₀N₇O₃: 418.16276. Observed 418.16178.

2-[5-(5-carbamimidoyl-pyridin-2-yl)-2-methoxyphenyl]-1H-benzimidazole-5-carboxamidine acetate salt (36). The same procedure described for compound 4 was used, starting with compound 35. Yield 80%, mp 231-232° C^{dec}. ¹H NMR (D₂O/DMSO-d₆); δ 1.83 (s, 3xCH₃), 4.18 (s, 3H), 7.42 (d, J=8.4 Hz, 1H), 7.63 (d, J=8.4 Hz, 1H), 7.78-7.88 (m, 2H), 8.10-8.33 (m, 3H), 9.00 (s, 1H), 9.17 (s, 1H). MS (m/z, rel.int.), 385 (M⁺, 4), 351 (100), 337 (75). Anal. (C₂₁H₁₉N₇O-3.0CH₃CO₂H-1.5H₂O): C, H, N.

### Example 10

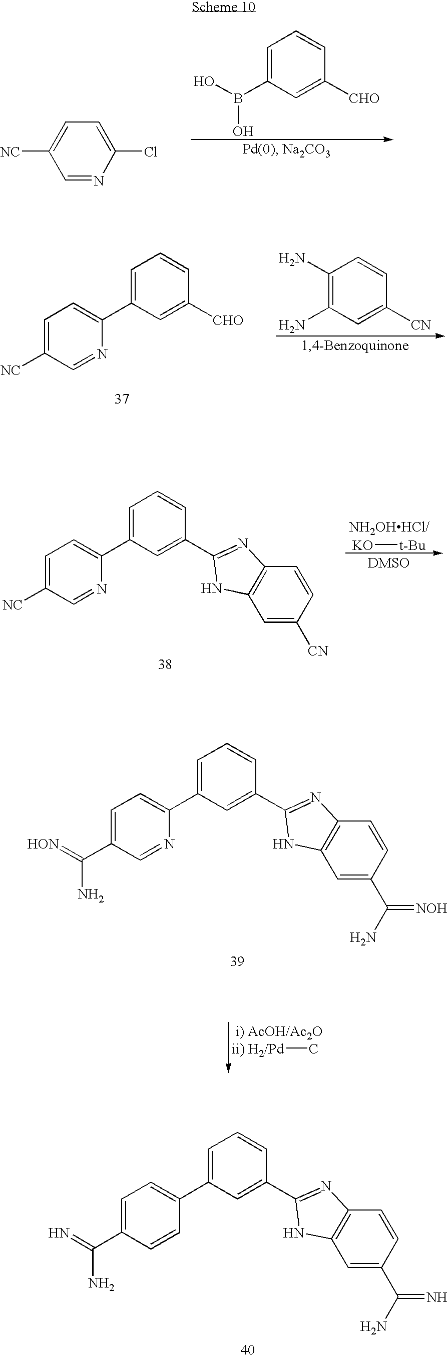

6-(3-formylphenyl)-nicotinonitrile (37). Referring now to Scheme 10, the same procedure described for compound 33 was used, employing 3-formylphenyl boronic acid instead of 3-formyl-4-methoxy-phenyl boronic acid.

Yield 58%; mp 182-183° C. ¹H NMR (DMSO-d₆); δ 7.77 (t, J=7.8 Hz, 1H), 8.04 (d, J=7.8 Hz, 1H), 8.29 (d, J=8.4 Hz, 1H), 8.43 (dd, J=8.4, 2.1 Hz, 1H), 8.48 (d, J=7.8 Hz, 1H), 8.69 (s, 1H), 9.13 (d, J=2.1 Hz, 1H), 10.13 (s, 1H). ¹³C NMR; δ 192.8, 157.7, 152.4, 141.1, 137.6, 136.8, 132.7, 130.7, 129.8, 128.3, 120.4, 116.9, 107.9, MS (m/z. rel.int.); 208 (M⁺, 100), 179 (70), 152 (15), 125 (5). High resolution mass calcd. for C₁₃H₈N₂O: 208.06366. Observed 208.06066.

2-[3-(5-cyanopyridin-2-yl)-phenyl]-1H-benzimidazole-5-carbonitrile (38). The same procedure described for compound 2 was used, starting with compound 37. Yield 83.5%, mp 281-282° C. ¹H NMR (DMSO-d₆); δ 7.63 (d, J=7.8 Hz, 1H), 7.76 (t, J=7.8 Hz, 1H), 7.81-7.88 (m, 1H), 8.24-8.38 (m, 4H), 8.49 (d, J=7.8 Hz, 1H), 9.03 (s, 1H), 9.18 (s, 1H), 13.61 (brs, 1H). ¹³C NMR; δ 158.2, 152.5, 141.1, 137.6, 129.8, 129.2, 128.7, 126.0, 125.5, 124.0, 120.3, 119.9, 117.1, 112.9, 107.8, 104.0. MS (m/z, rel.int.); 321 (M⁺, 100), 293 (12), 268 (5). HRMS calcd. for C₂₀H₁₁N₅: 321.10145. Observed 321.10069.

N-hydroxy-2-{3-[5-(N-hydroxycarbamimidoyl)-pyridin-2-yl]-phenyl}-1H-benzimidazole-5-carboxamidine (39). The same procedure described for compound 3 was used, starting with compound 38. Yield 97%, mp 295-297° C.^{dec}. ¹H NMR (DMSO-d₆); 5.82 (s, 2H), 6.08 (s, 2H), 7.51-7.72 (m, 3H), 8.00 (s, 1H), 8.11-8.28 (m, 4H), 8.95 (s, 1H), 9.03 (s, 1H), 9.61 (s, 1H), 9.93 (s, 1H), 13.18 (brs, 1H). Anal. (C₂₀H₁₇N₇O₂.2.5H₂O)C, H, N.

2-[3-(5-Carbamimidoyl-pyridin-2-yl)-phenyl]-1H-benzimidazole-5-carboxamidine acetate salt (40). The same procedure described for compound 4 was used, starting with compound 39. Yield 73%, mp 198-200° C.^{dec}. ¹H NMR (D₂O/DMSO-d₆); δ 1.80 (s, 2.8xCH₃), 7.64 (d, J=8.1 Hz, 1H), 7.72-7.81 (m, 2H), 8.14 (s, 1H), 8.24-8.40 (m, 4H), 9.08 (s, 1H), 9.12 (s, 1H). MS (m/z, rel.int., EI/isobutane), 356 (M⁺+1, 5), 322 (100), 297 (5). Anal. (C₂₀H₁₇N₇.2.8CH₃CO₂H-1.0H₂O)C, H, N.

### Example 11

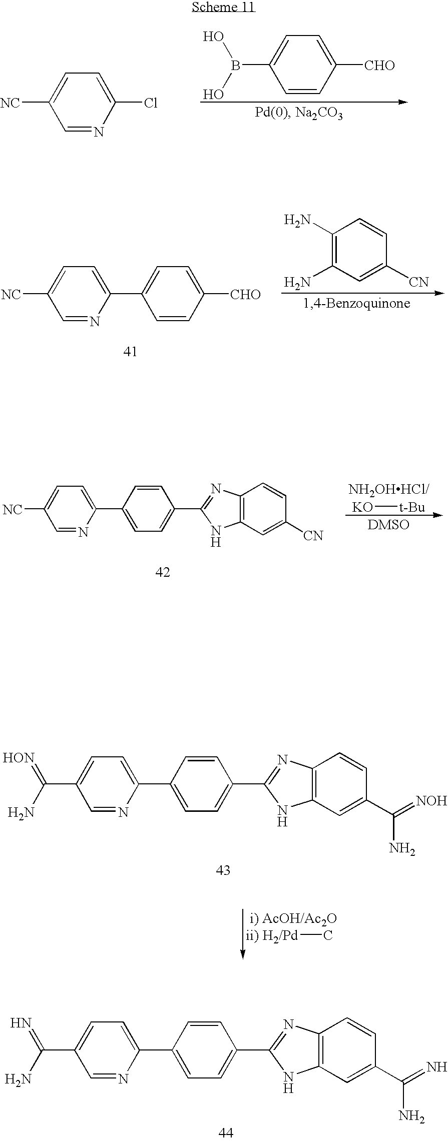

6-(4-Formylphenyl)-nicotinonitrile (41). Referring now to Scheme 11, the same procedure described for compound 33 was used, employing 4-formylphenyl boronic acid instead of 3-formyl-4-methoxy-phenyl boronic acid.

Yield 82%, mp 200-201° C. ¹H NMR (DMSO-d₆); δ 8.07 (d, J=8.1 Hz, 2H), 8.33 (d, J=8.4 Hz, 1H), 8.40 (d, J=8.1 Hz, 2H), 8.47 (dd, J=8.4, 2.1 Hz, 1H), 9.16 (d, J=2.1 Hz, 1H), 10.11 (s, 1H). ¹³C NMR (DMSO-d₆); δ 192.8, 157.7, 152.6, 142.0, 141.2, 137.1, 130.0, 127.9, 121.1, 117.0, 108.3. MS (m/z, rel.int.); 208 (M+, 85), 207 (100), 179 (55), 152 (15). Anal. (C₁₃H₈N₂O)C, H.

2-[4-(5-cyanopyridin-2-yl)-phenyl]-1H-benzimidazole-5-carbonitrile (42). The same procedure described for compound 2 was used, starting with compound 41. Yield 91%, mp 346-347° C. ¹H NMR (DMSO-d₆); δ 7.61 (d, J=7.8 Hz, 1H), 7.79 (d, J=7.8 Hz, 1H), 8.19 (s, 1H), 8.32 (d, J=8.4 Hz, 1H), 8.33-8.41 (m, 4H), 8.43 (dd, J=8.4, 2.1 Hz, 1H), 9.17 (d, J=2.1 Hz, 1H), 13.60 (brs, 1H). ¹³C NMR; δ 158.0, 152.6, 141.1, 138.6, 130.8, 127.8, 127.4, 120.5, 119.9, 117.2, 107.8, 104.2. MS (m/z, rel.int.); 321 (M⁺, 100), 293 (5), 204 (5), 160 (10). High resolution mass calcd. for C₂₀H₁₁N₅: 321.10145. Observed 321.10151.

N-hydroxy-2-{4-[5-(N-hydroxycarbamimidoyl)-pyridin-2-yl]-phenyl}-1H-benzimidazole-5-carboxamidine (43). The same procedure described for compound 3 was used, starting with compound 42. Yield 96%, mp 305-308° C.^{dec}. ¹H NMR (DMSO-d₆); 5.82 (s, 2H), 6.07 (s, 2H), 7.50-7.65 (m, 2H), 7.85 (s, 1H), 8.10-8.20 (m, 2H), 8.38 (s, 4H), 9.02 (s, 1H), 9.60 (s, 1H), 9.97 (s, 1H), 13.16 (brs, 1H). MS (m/z, rel.int.); 388 (M⁺+1, 100), 194 (40).

2-[4-(5-carbamimidoyl-pyridin-2-yl)-phenyl]-1H-benzimidazole-5-carboxamidine acetate salt (44). The same procedure described for compound 4 was used, starting with compound 43. Yield 82%, mp 240-241° C. ¹H NMR (D₂O/DMSO-d₆); δ 1.80 (s, 2.7xCH₃), 7.58 (d, J=8.4 Hz, 1H), 7.74 (d, J=8.4 Hz, 1H), 8.11 (s, 1H), 8.28-8.42 (m, 6H), 9.08 (s, 1H). Anal. (C₂₀H₁₇N₇-2.7CH₃CO₂H-1.3H₂O)C, H, N.

### Example 12

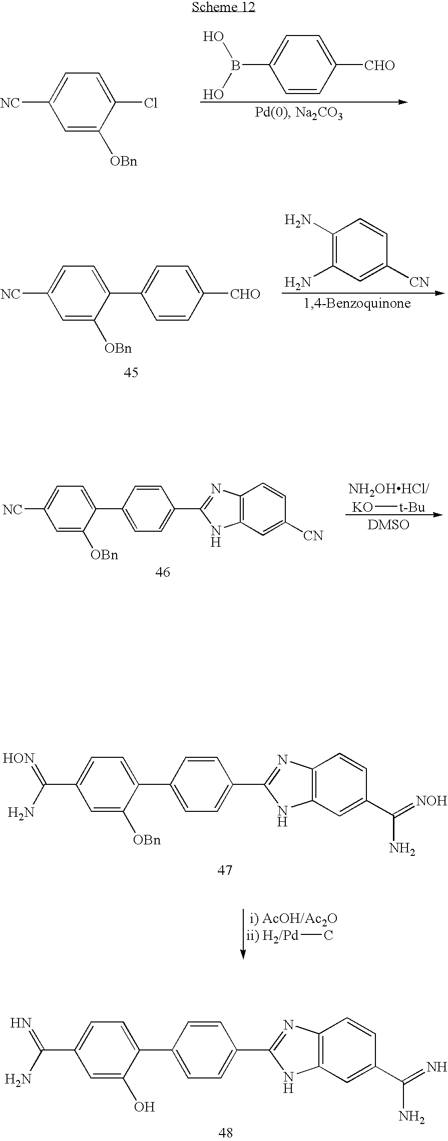

2-benzyloxy-4′-formylbiphenyl-4-carbonitrile (45). Referring now to Scheme 12, the same procedure described for compound 29 was used, employing 3-benzyloxy-4-bromobenzonitrile instead of 4-bromo-2-fluorobenzonitrile. Yield 69%, mp 131-132° C. ¹H NMR (DMSO-d₆); δ 5.24 (s, 2H), 7.30-7.39 (m, 5H), 7.54-7.61 (m, 2H), 7.76 (s, 1H), 7.80 (d, J=8.4 Hz, 2H), 7.96 (d, J=8.4 Hz, 2H), 10.04 (s, 1H). ¹³C NMR (DMSO-d₆); δ 192.7, 155.3, 142.3, 136.1, 135.3, 133.8, 131.5, 130.0, 129.1, 128.4, 127.9, 127.4, 125.1, 118.5, 116.5, 111.9, 70.2. MS (m/z, rel.int.); 313 (M⁺, 70), 285 (5), 220 (10), 193 (30), 164 (50), 91 (100).

2-(2′-benzyloxy-4′-cyanobiphenyl-4-yl)-1H-benzimidazole-5-carbonitrile (46). The same procedure described for compound 2 was used, starting with compound 45. Yield 75%, mp 205-208° C.^{dec}. ¹H NMR (DMSO-d₆); δ 5.24 (s, 2H), 7.31-7.43 (m, 6H), 7.56-7.64 (m, 3H), 7.70-7.81 (m, 4H), 8.23-8.26 (m, 2H), 13.57 (brs, 1H). MS (m/z, rel.int.); 427 (M⁺+1, 75), 371 (10), 293 (15), 241 (100).

2-[2′-benzyloxy-4′-(N-hydroxycarbamimidoyl)-biphenyl-4-yl]-N-hydroxy-1H-benzimidazole-5-carboxamidine (47). The same procedure described for compound 3 was used, starting with compound 46. Yield 90%, mp 250-253° C.^{dec}. ¹H NMR (DMSO-d₆); 5.21 (s, 2H), 5.83 (s, 2H), 5.93 (s, 2H), 7.28-7.43 (m, 7H), 7.48-7.62 (m, 4H), 7.75-7.80 (m, 2H), 8.16-8.21 (m, 2H), 9.59 (s, 1H), 9.73 (s, 1H), 13.00 (brs, 1H). MS (m/z, rel.int.); 493 (M⁺+1, 45), 247 (100).

2-(4′-carbamimidoyl-2′-hydroxy-biphenyl-4-yl)-1H-benzimidazole-5-carboxamidine acetate salt (48). The same procedure described for compound 4 was used, starting with compound 47. Yield 73%, mp 220-222° C. ¹H NMR (D₂O/DMSO-d₆); δ 1.82 (s, 3xCH₃), 7.30-7.50 (m, 3H), 7.60-7.78 (m, 2H), 7.85 (m, 2H), 8.20-8.38 (m, 3H). MS (m/z, rel.int.); 371 (M⁺+1, 80), 186 (100). Anal. (C₂₁H₁₈N₆O-3.0CH₃CO₂H-0.4H₂O-1.0C₂H₅OH)C, H, N.

### Example 13

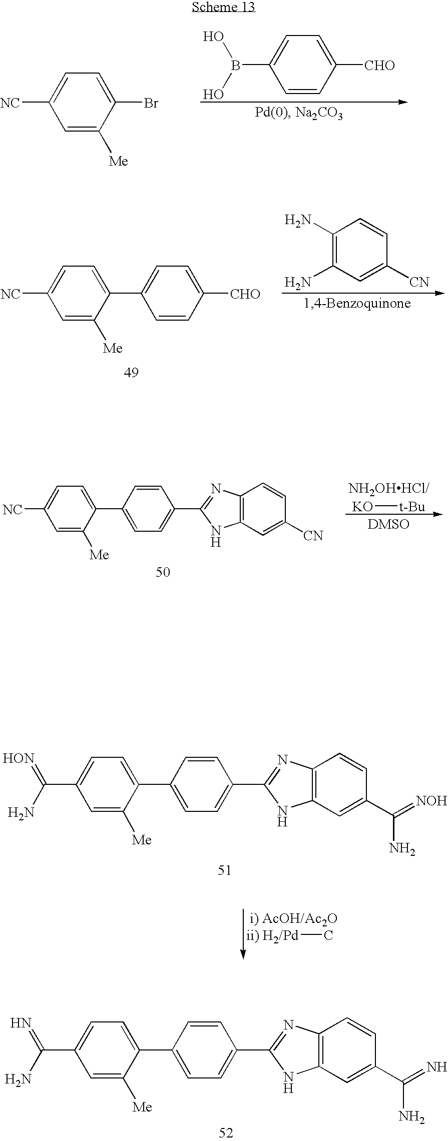

4′-formyl-2-methyl-biphenyl-4-carbonitrile (49). Referring now to Scheme 13, the same procedure described for compound 29 was used, employing 4-bromo-3-methyl-benzonitrile instead of 4-bromo-2-fluorobenzonitrile. Yield 71%, mp 130-130.5° C. ¹H NMR (DMSO-d₆); δ 2.28 (s, 3H), 7.46 (d, J=7.8 Hz, 1H), 7.63 (d, J=8.1 Hz, 2H), 7.77 (d, J=7.8 Hz, 1H), 7.86 (s, 1H), 8.02 (d, J=8.1 Hz, 2H), 10.08 (s, 1H). ¹³C NMR (DMSO-d₆); δ 192.7, 145.4, 144.9, 136.6, 135.3, 133.9, 130.3, 129.7, 129.6, 129.5, 118.6, 110.7, 19.6. MS(m/z, rel.int.); 221 (M⁺, 100), 203 (5), 192 (40), 177 (20), 165 (25).

2-(4′-cyano-2′-methyl-biphenyl-4-yl)-1H-benzimidazole-5-carbonitrile (50). The same procedure described for compound 2 was used, starting with compound 49. Yield 74%. mp 269-270° C. ¹H NMR (DMSO-d₆); δ 2.34 (s, 3H), 7.49 (d, J=7.8 Hz, 1H), 7.60-7.63 (m, 3H), 7.77 (d, J=7.8 Hz, 2H), 7.85 (s, 1H), 8.16-8.32 (m, 3H), 13.57 (brs, 1H). ¹³C NMR (DMSO-d₆); 145.1, 141.6, 136.7, 133.8, 130.4, 129.7, 129.5, 128.5, 126.8, 119.9, 118.7, 110.3, 19.7. MS (m/z, rel.int.); 335 (M⁺+1, 100), 306 (5), 176 (35).

N-hydroxy-2-[4′-(N-hydroxycarbamimidoyl)-2′-methyl-biphenyl-4-yl]-1H-benzimidazole-5-carboxamidine (51). The same procedure described for compound 3 was used, starting with compound 50. Yield 93%, mp 300-302° C. ¹H NMR (DMSO-d₆); 2.33 (s, 3H), 5.84 (s, 4H), 7.29 (d, J=7.8 Hz, 1H), 7.53-7.66 (m, 5H), 7.82 (s, 1H), 7.99 (s, 1H), 8.26 (d, J=7.8 Hz, 2H), 9.59 (s, 1H). 9.66 (s, 1H), 13.02 (s, 1H). MS (m/z, rel.int.); 401 (M⁺+1, 100), 163 (25).

2-(4′-carbamimidoyl-2′-methylbiphenyl-4-yl)-1H-benzimidazole-5-carboxamidine acetate salt (52). The same procedure described for compound 4 was used, starting with compound 51. Yield 82%, mp 193-195° C. ¹H NMR (D₂O/DMSO-d₆); δ 1.82 (s, 3xCH₃), 2.40 (s, 3H), 7.60-8.00 (m, 7H), 8.20 (s, 1H), 8.25-8.38 (m, 2H). Anal. (C₂₂H₂₀N₆-3.0CH₃CO₂H-0.25H₂O)C, H, N.

### Example 14

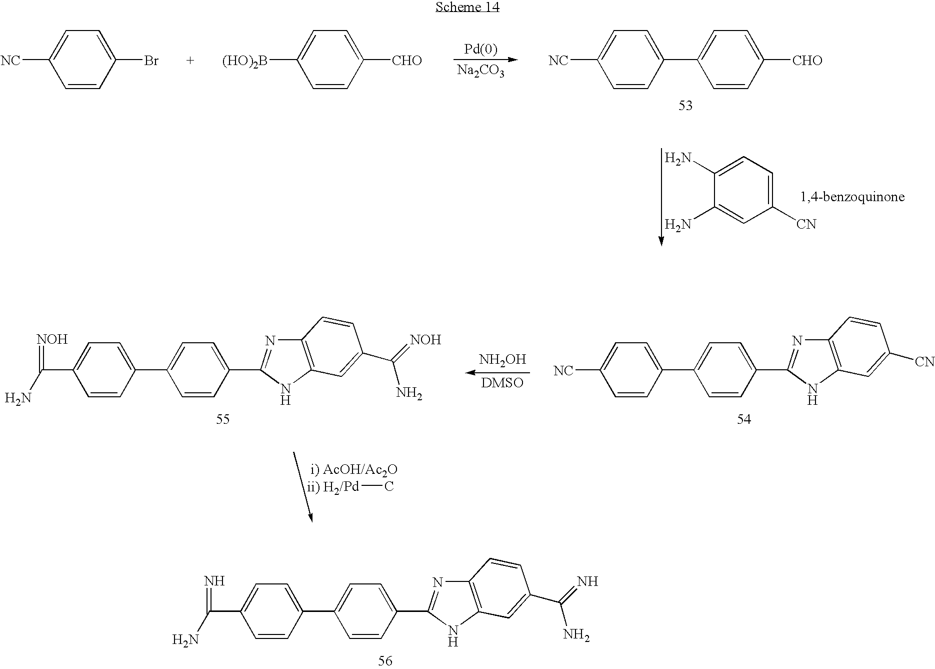

### Example 15

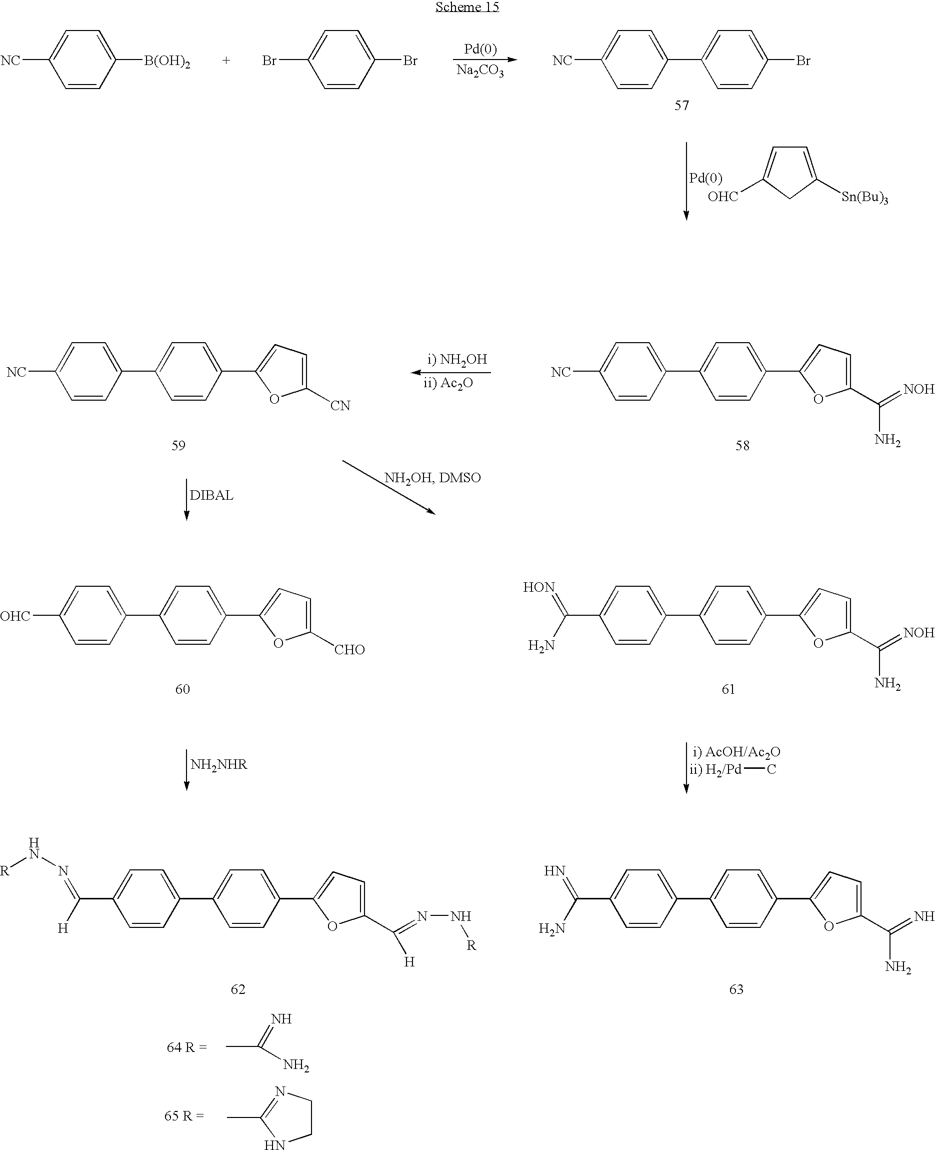

### Example 16

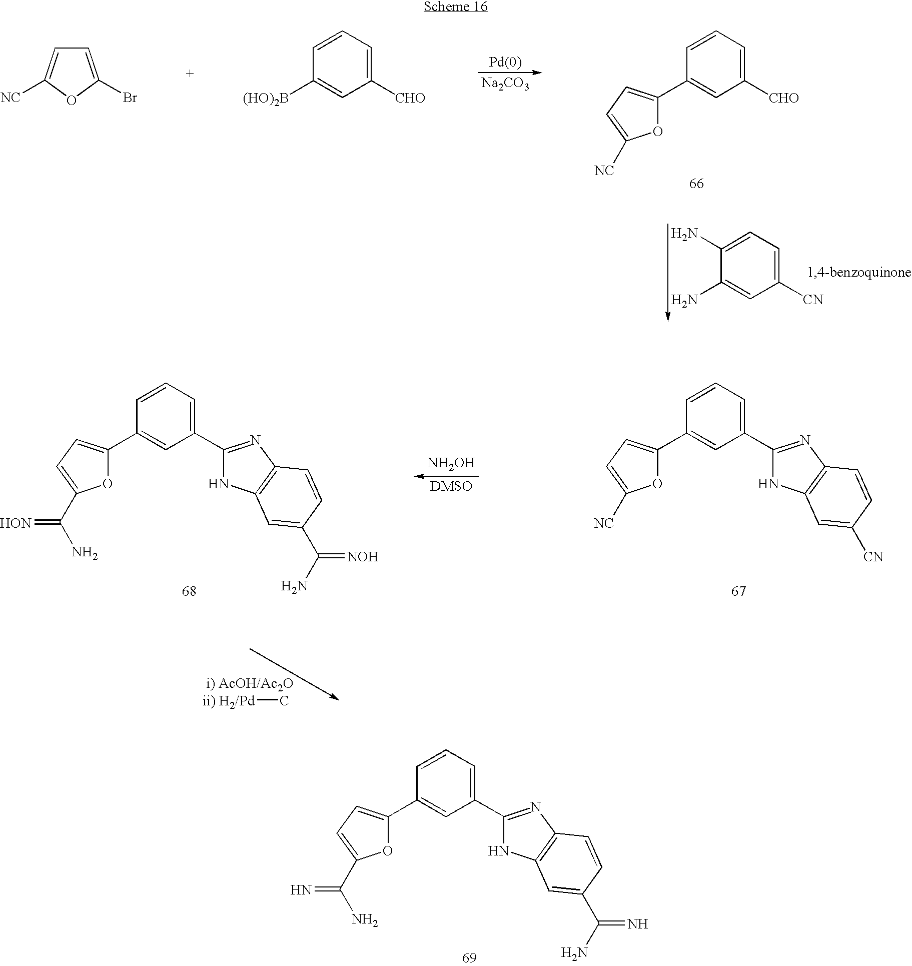

### Example 17

Tables 1 and 2 show potent in vitro data for certain compounds of Formula (I) and Formula (II). Eight compounds (24, 40, 36, 16, 56, 52, 63, and 70 show IC-50 values versus Trypanosoma brucei rhodesiense (T.b.r.) at less than 20 nM. Five compounds (4, 56, 32, 63, and 70) show IC-50 values versus Plasmodium falciparum (p.f.) at less than 3 nM. Compounds 16 and 40 cure the virulent STIP900 strain of T.b.r. in a mouse model. The compounds can thus be employed as treatments of second-stage human African trypanosomiasis.

TABLE 1
In vitro Activities of Dicationic molecules vs. T. b. rhodesiense and P. falciparum.

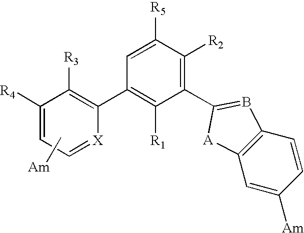

| Compound | | | | | | | | | | T.b.r. | P.f. |
|---|---|---|---|---|---|---|---|---|---|---|---|
| No. | Am | A | B | X | R₁ | R₂ | R₃ | R₄ | R₅ | IC50 nM | IC50 nM |
| 24 | p-Am | NH | N | CH | H | H | H | H | H | 4.4 | 27.5 |
| 40 | p-Am | NH | N | N | H | H | H | H | H | 15 | 96 |
| 20 | p-Am | NH | N | CH | H | OMe | H | H | H | 53 | 40 |
| 36 | p-Am | NH | N | N | H | OMe | H | H | H | 14 | 364 |
| 16 | p-Am | NH | N | CH | OH | H | H | H | H | 17.0 | 131 |
| 4 | p-Am | NH | N | CH | H | OH | H | H | H | 27 | 2.1 |
| 8 | m-Am | NH | N | CH | H | OH | H | H | H | 213 | 34 |
| 3, salt | p-AmOH | NH | N | CH | H | OH | H | H | H | >17K | >170K |
| 12 | p-Am | NH | N | CH | H | OH | H | H | OMe | 31 | 131 |
| 28 | p-Am | NH | N | CH | H | H | Me | H | H | 23 | 55 |
| 69 | a) | NH | N | CH | H | H | H | H | H | | |

a) the benzamidine is replaced by a 5-amidinofuran-2-yl

TABLE 2
In vitro Activities of Dicationic molecules vs. T. b. rhodesiense
and P. falciparum.

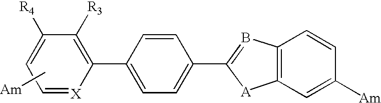

| | | | | | | | T.b.r. | P.f. |
|---|---|---|---|---|---|---|---|---|
| Compound | | | | | | | IC50 | IC50 |
| No. | Am | A | B | X | R₄ | R₃ | nM | nM |
| 56 | p | NH | N | CH | H | H | 7.7 | 0.5 |
| 44 | p | NH | N | N | H | H | 41 | 7 |
| 32 | p | NH | N | CH | F | H | 35 | 1.3 |
| 48 | p | NH | N | CH | H | OH | 27 | 19.1 |
| 52 | p | NH | N | CH | H | CH₃ | 6.0 | |
| 64^{c} | p | NA | NA | CH | NA | NA | 2.3K | 42.2 |
| 65^{b} | p | NA | NA | CH | NA | NA | 345 | |
| 63^{a} | p | NA | NA | CH | NA | NA | 5.2 | 1.1 |
| 70^{d} | p | NA | NA | CH | H | H | 5.0 | 1.0 |

^{a)}5-furan amidine replaces benzimidazole amidine;
^{b)}furan replaces benzimidazole and imidoguanylhydrazones replace amidines;
^{c)}furan replaces benzimidazole and guanylhydrazones replace amidines;
^{d)}phenyl replaces benzimidazole.

It will be understood that various details of the presently disclosed subject matter can be changed without departing from the scope of the presently disclosed subject matter. Furthermore, the foregoing description is for the purpose of illustration only, and not for the purpose of limitation.

1. A compound comprising a diaryl ring structure of Formula (I): 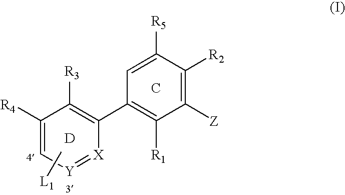
wherein:
X and Y are each independently selected from the group consisting of CH, N, O and S, and wherein Y can be present or absent;
R₁ is selected from the group consisting of H, alkyl, halo, alkoxyl, aryloxyl, and aralkoxyl;
R₂, R₃, R₄ and R₅ are each independently selected from the group consisting of H, alkyl, halo, hydroxyl, alkoxyl, aryloxyl, and aralkoxyl;
Z is selected from one of: 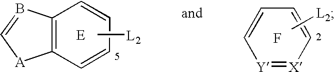
wherein:
A is selected from the group consisting of O, S, and NR₆, and wherein R₆ is selected from one of H and alkyl;
B is selected from the group consisting of O, S, and N;
X′ and Y′ are each independently selected from the group consisting of CH, N, O and S, and Y′ can be present or absent;
L₁ and L₂ are each independently selected from the group consisting of: 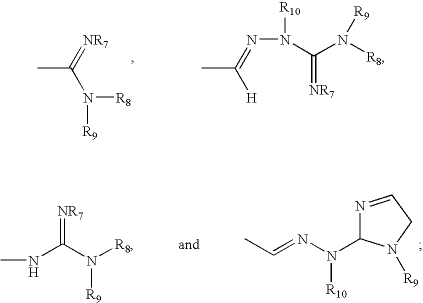
wherein:
L₁ is at one of the 3′-position and 4′-position of the diaryl ring D;
R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
R₇ and R₈ together represent a C₂ to C₁₀ alkyl, hydroxyalkyl, or alkylene; or
R₇ and R₈ together are: 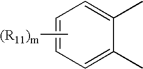
wherein m is an integer from 1 to 3, and R₁₁ is selected from one of H and —CONHR₁₂NR₁₃R₁₄, wherein:
R₁₂ is alkyl, and R₁₃ and R₁₄ are each independently selected from one of H and alkyl; or
a pharmaceutically acceptable salt thereof.

2. The compound of claim 1, wherein:
X is selected from one of CH and N;
Y is present and is CH;
Z is 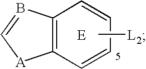
wherein:
A is NH;
B is N; and
L₂ is at the 5-position of ring E;
L₁ and L₂ are each independently 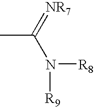
wherein R₇ is selected from one of H and hydroxyl; and
R₈ and R₉ are each H;
R₁ and R₄ are each H;
R₂ is selected from the group consisting of H, hydroxyl, and alkoxyl;
R₃ is selected from one of H and alkyl; and
R₅ is selected from one of H and alkoxyl.

3. The compound of claim 2, wherein
X is CH;
R₂ is selected from the group consisting of H, hydroxyl, and alkoxyl;
R₃ is selected from one of H and alkyl;
R₅ is selected from one of H and alkoxyl; and
R₇ is selected from one of H and hydroxyl.

4. The compound of claim 3, wherein R₂, R₃, and R₅ are each H.

5. The compound of claim 3, wherein R₂ is hydroxyl.

6. The compound of claim 3, wherein at least one of R₂ and R₅ is alkoxyl.

7. The compound of claim 3, wherein R₃ is alkyl.

8. The compound of claim 3, wherein R₇ is H.

9. The compound of claim 3, wherein R₇ is hydroxyl.

10. The compound of claim 3, wherein L₁ is at the 4′-position of the diaryl ring D.

11. The compound of claim 3, wherein L₁ is at the 3′-position of the diaryl ring D.

12. The compound of claim 2, wherein
X is N;
R₃ and R₅ are each H;
R₂ is selected from one of H and alkoxyl; and
R₇ is selected from one of H and hydroxyl.

13. The compound of claim 12, wherein R₂ is H.

14. The compound of claim 12, wherein R₂ is alkoxyl.

15. The compound of claim 12, wherein R₇ is H.

16. The compound of claim 12, wherein R₇ is OH.

17. The compound of claim 12, wherein L₁ is in the 4′-position of the diaryl ring D.

18. The compound of claim 1, wherein
X is O;
Y is absent;
Z is 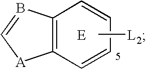
wherein A is NH;
B is N; and
L₂ is at the 5-position of ring E;
L₁ and L₂ are each independently 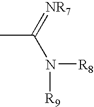
wherein R₇ is selected from one of H and hydroxyl; and
R₈ and R₉ are each H; and
R₁, R₂, R₃, R₄, and R₅ are each H.

19. The compound of claim 18, wherein R₇ is H.

20. The compound of claim 18, wherein R₇ is hydroxyl.

21. The compound of claim 1, wherein the compound is selected from the group consisting of:
N-hydroxy-2-[4-hydroxy-4′-(N-hydroxycarbamimidoyl)-biphenyl-3-yl]-1H-benzimidazole-5-carboxamidine;
2-(4′-carbamimidoyl-4-hydroxy-biphenyl-3-yl)-1H-benzimidazole-5-carboxamidine;
N-hydroxy-2-[4-hydroxy-3′-(N-hydroxycarbamimidoyl)-biphenyl-3-yl]-1H-benzimidazol-5-carboxamidine;
2-(3′-carbamimidoyl-4-hydroxy-biphenyl-3-yl)-1H-benzimidazole-5-carboxamidine;
N-hydroxy-2-[4-hydroxy-4-(N-hydroxycarbamimidoyl)-5-methoxy-biphenyl-3-yl] 1H-benzimidazole-5-carboxamidine;
2-(4′-carbamimidoyl-4-hydroxy-5-methoxy-biphenyl-3-yl)-1H-benzimidazole-5-carboxamidine;
N-hydroxy-2-[4′-(N-hydroxycarbamimidoyl)-4-methoxy-biphenyl-3-yl]-1H-benzimidazole-5-carboxamidine;
2-(4′-carbamimidoyl-4-methoxy-biphenyl-3-yl)-1H-benzimidazole-5-carboxamidine;
N-hydroxy-2-[4′-(N-hydroxycarbamimidoyl)-biphenyl-3-yl]-1H-benzimidazole-5-carboxamidine;
2-(4′-carbamimidoyl-biphenyl-3-yl)-1H-benzimidazole-5-carboxamidine;
N-hydroxy-2-[4′-(N-hydroxycarbamimidoyl)-2′-methyl-biphenyl-3-yl]-1H-benzimidazole-5-carboxamidine;
2-(4′-carbamimidoyl-2′-methyl-biphenyl-3-yl)-1H-benzimidazole-5-carboxamidine;
N-hydroxy-2-{5-[5-(N-hydroxycarbamimidloyl)-pyridin-2-yl]-2-methoxy-phenyl}-1H-benzimidazole-5-carboxamidine;
2-[5-(5-carbamimidoyl-pyridin-2-yl)-2-methoxyphenyl]-1H-benzimidazole-5-carboxamidine;
N-hydroxy-2-{3-[5-(N-hydroxycarbamimidoyl)-pyridin-2-yl]-phenyl})-1H-benzimidazole-5-carboxamidine; and
2-[3-(5-Carbamimidoyl-pyridin-2-yl)-phenyl]-1H-benzimidazole-5-carboxamidine.

22. The compound of claim 1, wherein the compound of Formula (I) has the following structure:

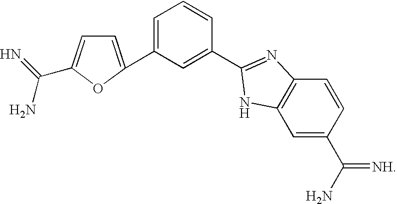

23. The compound of claim 1, wherein the compound comprises a pharmaceutically acceptable salt.

24. The pharmaceutically acceptable salt of claim 23, wherein the pharmaceutically acceptable salt comprises an acetate salt.

25. A pharmaceutical formulation comprising:
(a) a pharmaceutically acceptable carrier; and
(b) a compound comprising a diaryl ring structure of Formula (I): 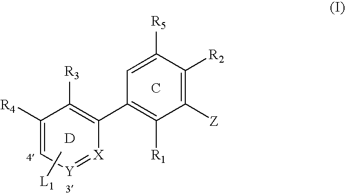
wherein:
X and Y are each independently selected from the group consisting of CH, N, O and S, and wherein Y can be present or absent;
R₁, R₂, R₃, R₄ and R₅ are each independently selected from the group consisting of H, alkyl, halo, hydroxyl, alkoxyl, aryloxyl, and aralkoxyl;
Z is selected from one of: 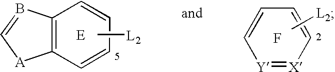
wherein:
A is selected from the group consisting of O, S, and NR₆, and wherein R₆ is selected from one of H and alkyl;
B is selected from the group consisting of O, S, and N;
X′ and Y′ are each independently selected from the group consisting of CH, N, O and S, and Y′ can be present or absent;
L₁ and L₂ are each independently selected from the group consisting of: 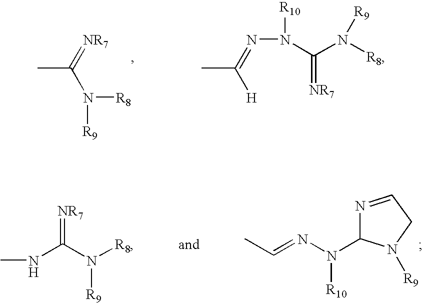
wherein:
L₁ is at one of the 3′-position and 4′-position of the diaryl ring D;
R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
R₇ and R₈ together represent a C₂ to C₁₀ alkyl, hydroxyalkyl, or alkylene; or
R₇ and R₈ together are: 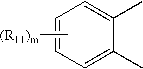
wherein m is an integer from 1 to 3, and R₁, is selected from one of H and —CONHR₁₂NR₁₃R₁₄, wherein:
R₁₂ is alkyl, and R₁₃ and R₁₄ are each independently selected from one of H and alkyl; or
a pharmaceutically acceptable salt thereof.

26. The pharmaceutical formulation of claim 25, wherein:
X is selected from one of CH and N;
Y is present and is CH;
Z is 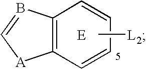
wherein:
A is NH;
B is N; and
L₂ is at the 5-position of ring E;
L₁ and L₂ are each independently 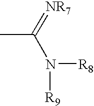
wherein R₇ is selected from one of H and hydroxyl; and
R₈ and R₉ are each H;
R₁ is selected from one of H and hydroxyl;
R₂ is selected from the group consisting of H, hydroxyl, and alkoxyl;
R₃ is selected from one of H and alkyl;
R₄ is H; and
R₅ is selected from one of H and alkoxyl.

27. The pharmaceutical formulation of claim 26, wherein
X is CH;
R₁ is selected from one of H and hydroxyl;
R₂ is selected from the group consisting of H, hydroxyl, and alkoxyl;
R₃ is selected from one of H and alkyl;
R₄ is H;
R₅ is selected from one of H and alkoxyl; and
R₇ is selected from one of H and hydroxyl.

28. The pharmaceutical formulation of claim 27, wherein R₂, R₃, and R₅ are each H.

29. The pharmaceutical formulation of claim 27, wherein at least one of R₁ and R₂ is hydroxyl.

30. The pharmaceutical formulation of claim 27, wherein at least one of R₂ and R₅ is alkoxyl.

31. The pharmaceutical formulation of claim 27, wherein R₃ is alkyl.

32. The pharmaceutical formulation of claim 27, wherein R₇ is H.

33. The pharmaceutical formulation of claim 27, wherein R₇ is hydroxyl.

34. The pharmaceutical formulation of claim 27, wherein L₁ is at the 4′-position of the diaryl ring D.

35. The pharmaceutical formulation of claim 27, wherein L₁ is at the 3′-position of the diaryl ring D.

36. The pharmaceutical formulation of claim 26, wherein
X is N;
R₁, R₃ and R₅ are each H;
R₂ is selected from one of H and alkoxyl; and
R₇ is selected from one of H and hydroxyl.

37. The pharmaceutical formulation of claim 36, wherein R₂ is H.

38. The pharmaceutical formulation of claim 36, wherein R₂ is alkoxyl.

39. The pharmaceutical formulation of claim 36, wherein R₇ is H.

40. The pharmaceutical formulation of claim 36, wherein R₇ is OH.

41. The pharmaceutical formulation of claim 36, wherein L₁ is in the 4′-position of the diaryl ring D.

42. The pharmaceutical formulation of claim 25, wherein
X is O;
Y is absent;
Z is 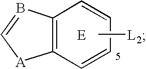
wherein A is NH;
B is N; and
L₂ is at the 5-position of ring E;
L₁ and L₂ are each independently 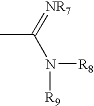
wherein R₇ is selected from one of H and hydroxyl; and
R₈ and R₉ are each H; and
R₁, R₂, R₃, R₄, and R₅ are each H.

43. The pharmaceutical formulation of claim 42, wherein R₇ is H.

44. The pharmaceutical formulation of claim 42, wherein R₇ is hydroxyl.

45. The pharmaceutical formulation of claim 25, wherein the compound is selected from the group consisting of:
2-(4′-carbamimidoyl-biphenyl-3-yl)-1H-benzimidazole-5-carboxamidine;
2-[3-(5-carbamimidoyl-pyridin-2-yl)-phenyl]-1H-benzimidazole-5-carboxamidine;
N-hydroxy-2-[4-hydroxy-4′-(N-hydroxycarbamimidoyl)-biphenyl-3-yl]-1H-benzimidazole-5-carboxamidine;
2-(4′-carbamimidoyl-4-hydroxy-biphenyl-3-yl)-1H-benzimidazole-5-carboxamidine;
2-(3′-carbamimidoyl-4-hydroxy-biphenyl-3-yl)-1H-benzimidazole-5-carboxamidine;
2-(4′-carbamimidoyl-4-hydroxy-5-methoxy-biphenyl-3-yl)-1H-benzimidazole-5-carboxamidine;
2-(4′-carbamimidoyl-4-methoxy-biphenyl-3-yl)-1H-benzimidazole-5-carboxamidine;
2-(4′-carbamimidoyl-2′-methyl-biphenyl-3-yl)-1H-benzimidazole-5-carboxamidine;
2-[5-(5-carbamimidoyl-pyridin-2-yl)-2-methoxyphenyl]-1H-benzimidazole-5-carboxamidine;
2-(4′-carbamimidoyl-2-hydroxy-biphenyl-3-yl)-1H-benzimidazole-5-carboxamidine.

46. The pharmaceutical formulation of claim 25, wherein the compound of Formula (I) has the following structure:

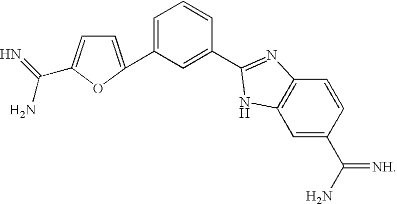

47. The pharmaceutical formulation of claim 25, wherein the compound comprises a pharmaceutically acceptable salt.

48. The pharmaceutically acceptable salt of claim 47, wherein the pharmaceutically acceptable salt comprises an acetate salt.

49. A method of treating microbial infection in a subject in need thereof, the method comprising administering to the subject an effective amount of a compound comprising a diary ring structure of Formula (I): 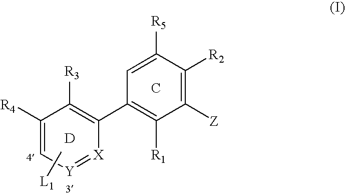
wherein:
X and Y are each independently selected from the group consisting of CH, N, O and S, and wherein Y can be present or absent;
R₁, R₂, R₃, R₄ and R₅ are each independently selected from the group consisting of H, alkyl, halo, hydroxyl, alkoxyl, aryloxyl, and aralkoxyl;
Z is selected from one of: 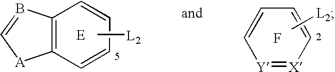
wherein:
A is selected from the group consisting of O, S, and NR₆, and wherein R₆ is selected from one of H and alkyl;
B is selected from the group consisting of O, S, and N;
X′ and Y′ are each independently selected from the group consisting of CH, N, O and S, and Y′ can be present or absent;
L₁ and L₂ are each independently selected from the group consisting of: 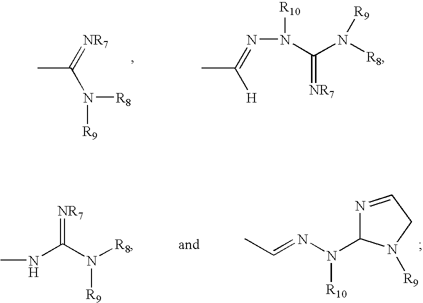
wherein:
L, is at one of the 3′-position and 4′-position of the diaryl ring D;
R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
R₇ and R₈ together represent a C₂ to C₁₀ alkyl, hydroxyalkyl, or alkylene; or
R₇ and R₈ together are: 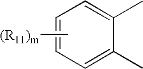
wherein m is an integer from 1 to 3, and R₁₁ is selected from one of H and —CONHR₁₂NR₁₃R₁₄, wherein:
R₁₂ is alkyl, and R₁₃ and R₁₄ are each independently selected from one of H and alkyl; or
a pharmaceutically acceptable salt thereof.

50. The method of claim 49, wherein:
X is selected from one of CH and N;
Y is present and is CH;
Z is 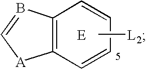
wherein:
A is NH;
B is N; and
L₂ is at the 5-position of ring E;
L₁ and L₂ are each independently 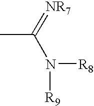
wherein R₇ is selected from one of H and hydroxyl; and
R₈ and R₉ are each H;
R₁ is selected from one of H and hydroxyl;
R₂ is selected from the group consisting of H, hydroxyl, and alkoxyl;
R₃ is selected from one of H and alkyl;
R₄ is H; and
R₅ is selected from one of H and alkoxyl.

51. The method of claim 50, wherein
X is CH;
R₁ is selected from one of H and hydroxyl;
R₂ is selected from the group consisting of H, hydroxyl, and alkoxyl;
R₃ is selected from one of H and alkyl;
R₄ is H;
R₅ is selected from one of H and alkoxyl; and
R₇ is selected from one of H and hydroxyl.

52. The method of claim 51, wherein R₂, R₃, and R₅ are each H.

53. The method of claim 51, wherein at least one of R₁ and R₂ is hydroxyl.

54. The method of claim 51, wherein at least one of R₂ and R₅ is alkoxyl.

55. The method of claim 51, wherein R₃ is alkyl.

56. The method of claim 51, wherein R₇ is H.

57. The method of claim 51, wherein R₇ is hydroxyl.

58. The method of claim 51, wherein L₁ is at the 4′-position of the diaryl ring D.

59. The method of claim 51, wherein L₁ is at the 3′-position of the diaryl ring D.

60. The method of claim 50, wherein
X is N;
R₁, R₃ and R₅ are each H;
R₂ is selected from one of H and alkoxyl; and
R₇ is selected from one of H and hydroxyl.

61. The method of claim 60, wherein R₂ is H.

62. The method of claim 60, wherein R₂ is alkoxyl.

63. The method of claim 60, wherein R₇ is H.

64. The method of claim 60, wherein R₇ is OH.

65. The method of claim 60, wherein L₁ is in the 4′-position of the diaryl ring D.

66. The method of claim 49, wherein
X is O;
Y is absent;
Z is 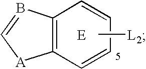
wherein A is NH;
B is N; and
L₂ is at the 5-position of ring E;
L₁ and L₂ are each independently 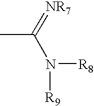
wherein R₇ is selected from one of H and hydroxyl; and
R₈ and R₉ are each H; and
R₁, R₂, R₃, R₄, and R₅ are each H.

67. The method of claim 66, wherein R₇ is H.

68. The method of claim 66, wherein R₇ is hydroxyl.

69. The method of claim 49, wherein the compound is selected from the group consisting of:
2-(4′-carbamimidoyl-biphenyl-3-yl)-1H-benzimidazole-5-carboxamidine;
2-[3-(5-carbamimidoyl-pyridin-2-yl)-phenyl]-1H-benzimidazole-5-carboxamidine;
N-hydroxy-2-[4-hydroxy-4′-(N-hydroxycarbamimidoyl)-biphenyl-3-yl]-1H-benzimidazole-5-carboxamidine;
2-(4′-carbamimidoyl-4-hydroxy-biphenyl-3-yl)-1H-benzimidazole-5-carboxamidine;
2-(3′-carbamimidoyl-4-hydroxy-biphenyl-3-yl)-1H-benzimidazole-5-carboxamidine;
2-(4′-carbamimidoyl-4-hydroxy-5-methoxy-biphenyl-3-yl)-1H-benzimidazole-5-carboxamidine;
2-(4′-carbamimidoyl-4-methoxy-biphenyl-3-yl)-1H-benzimidazole-5-carboxamidine;
2-(4′-carbamimidoyl-2′-methyl-biphenyl-3-yl)-1H-benzimidazole-5-carboxamidine;
2-[5-(5-carbamimidoyl-pyridin-2-yl)-2-methoxyphenyl]-1H-benzimidazole-5-carboxamidine; and
2-(4′-carbamimidoyl-2-hydroxy-biphenyl-3-yl)-1H-benzimidazole-5-carboxamidine.

70. The method of claim 49, wherein the compound comprises a pharmaceutically acceptable salt.

71. The pharmaceutically acceptable salt of claim 70, wherein the pharmaceutically acceptable salt comprises an acetate salt.

72. The method of claim 49, wherein the microbial infection is selected from one of a Trypanosoma brucei rhodesiense infection and a Plasmodium falciparum infection.

73. The method of claim 72, wherein the microbial infection comprises a Trypanosoma brucei rhodesiense infection.

74. The method of claim 72, wherein the microbial infection comprises a Plasmodium falciparum infection.

75. The method of claim 49, wherein the compound of Formula (I) has the following structure:

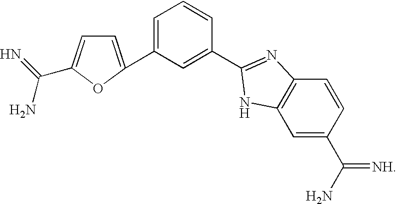

76. A compound comprising the diaryl ring structure of Formula (II): 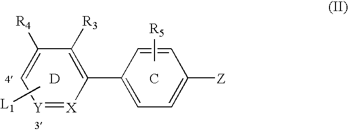
wherein:
X and Y are each independently selected from the group consisting of CH, N, O and S, and Y can be present or absent;
R₃, R₄, and R₅ are each independently selected from the group consisting of H, alkyl, halogen, hydroxyl, alkoxyl, aryloxyl, and aralkoxyl;
Z is selected from one of: 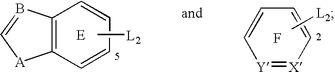
wherein:
A is selected from the group consisting of O, S, and NR₆, and wherein R₆ is selected from one of H and alkyl;
B is selected from the group consisting of O, S, and N;
X′ and Y′ are each independently selected from the group consisting of CH, N, O and S, and Y′ can be present or absent;
L₁ and L₂ are each independently selected from the group consisting of: 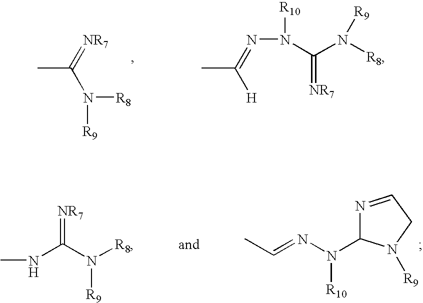
wherein:
L₁ is at one of the 3′-position and the 4′-position of the diary ring D;
R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
R₇ and R₈ together represent a C₂ to C₁₀ alkyl, hydroxyalkyl, or alkylene; or
R₇ and R₈together are: 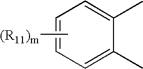
wherein:
m is an integer from 1 to 3, and R₁₁ is selected from one of H and —CONHR₁₂NR₁₃R₁₄, wherein R₁₂ is alkyl and R₁₃ and R₁₄ are each independently selected from one of H and alkyl; or
a pharmaceutically acceptable salt thereof.

77. The compound of claim 76, wherein:
X and Y are each CH;
R₃ is selected from the group consisting of H, alkyl, hydroxyl, alkoxyl, and araloxyl;
R₄ is selected from one of H and halogen;
R₅ is H;
Z is: 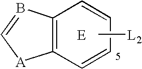
wherein:
A is NH;
B is N;
L₁ and L₂ are each independently: 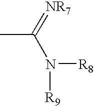
wherein:
R₇ is selected from one of H and hydroxyl; and
R₈ and R₉ are each H.

78. The compound of claim 77, wherein R₃ is H.

79. The compound of claim 77, wherein R₃ is alkyl.

80. The compound of claim 77, wherein R₃ is hydroxyl.

81. The compound of claim 77, wherein R₃ is araloxyl.

82. The compound of claim 77, wherein R₄ is H.

83. The compound of claim 77, wherein R₄ is halogen.

84. The compound of claim 77, wherein R₇ is H.

85. The compound of claim 77, wherein R₇ is hydroxyl.

86. The compound of claim 76, wherein:
X is N;
Y is CH;
R₃, R₄, and R₅ are each H;
Z is: 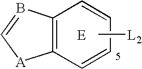
wherein:
A is NH;
B is N;
L₁ and L₂ are each independently: 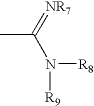
wherein:
L₁ is in the 4′-position of the diaryl ring D;
R₇ is selected from one of H and hydroxyl; and
R₈ and R₉ are each H.

87. The compound of claim 86, wherein R₇ is H.

88. The compound of claim 86, wherein R₇ is OH.

89. The compound of claim 76, wherein:
X and Y are each CH;
R₃, R₄, and R₅ are each H;
Z is: 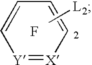
X′ is O
Y′ is absent;
L₁ and L₂ are each independently selected from the group consisting of: 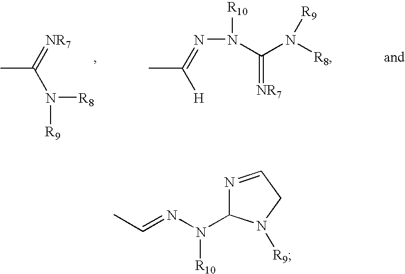
wherein:
R₇ is selected from one of H and OH; and
R₈, R₉ and R₁₀ are H.

90. The compound of claim 89, wherein L₁ and L₂ are each independently:

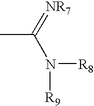

91. The compound of claim 90, wherein R₇ is H.

92. The compound of claim 90, wherein R₇ is OH.

93. The compound of claim 89, wherein L₁ and L₂ are each independently: 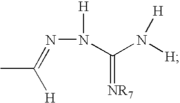
and R₇ is H.

94. The compound of claim 89, wherein L₁ and L₂ are each independently:

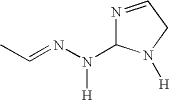

95. The compound of claim 76, wherein the compound is selected from the group consisting of:
2-[3-fluoro-4′-(N-hydroxycarbamimidoyl)-biphenyl-4-yl]-N-hydroxy-1H-benzimidazole-5-carboxamidine;
2-(4′-carbamimidoyl-3′-fluoro-biphenyl-4-yl)-1H-benzimidazole-5-carboxamidine;
N-hydroxy-2-{4-[5-(N-hydroxycarbamimidoyl)-pyridin-2-yl]-phenyl}-1H-benzimidazole-5-carboxamidine;
2-[4-(5-carbamimidoyl-pyridin-2-yl)-phenyl]-1H-benzimidazole-5-carboxamidine;
2-[2′-benzyloxy-4′-(N-hydroxycarbamimidoyl)-biphenyl-4-yl]-N-hydroxy-1H-benzimidazole-5-carboxamidine;
2-(4′-carbamimidoyl-2′-hydroxy-biphenyl-4-yl)-1H-benzimidazole-5-carboxamidine;
N-hydroxy-2-[4′-(N-hydroxycarbamimidoyl)-2′-methyl-biphenyl-4-yl]-1H-benzimidazole-5-carboxamidine; and
2-(4′-carbamimidoyl-2′-methyl-biphenyl-4-yl)-1H-benzimidazole-5-carboxamidine.

96. The compound of claim 76, wherein the compound is selected from the group of compounds having the following chemical structures:

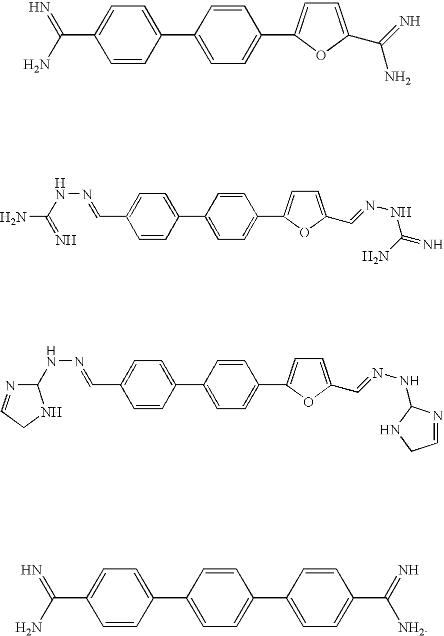

97. The compound of claim 76, wherein the compound comprises a pharmaceutically acceptable salt.

98. The pharmaceutically acceptable salt of claim 97, wherein the pharmaceutically acceptable salt comprises an acetate salt.

99. A pharmaceutical formulation comprising:
(a) a pharmaceutically acceptable carrier; and
(b) a compound comprising the diaryl ring structure of Formula (II): 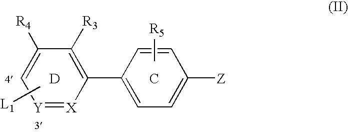
wherein:
X and Y are each independently selected from the group consisting of CH, N, O and S, and Y can be present or absent;
R₃, R₄, and R₅ are each independently selected from the group consisting of H, alkyl, halogen, hydroxyl, alkoxyl, aryloxyl, and aralkoxyl;
Z is selected from one of: 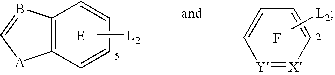
wherein:
A is selected from the group consisting of O, S, and NR₆, and wherein R₆ is selected from one of H and alkyl;
B is selected from the group consisting of O, S, and N;
X′ and Y′ are each independently selected from the group consisting of CH, N, O and S, and Y′ can be present or absent;
L₁ and L₂ are each independently selected from the group consisting of: 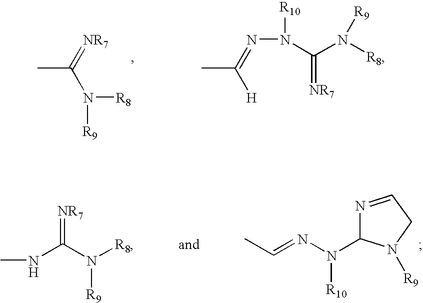
wherein:
L₁ is at one of the 3′-position and the 4′-position of the diary ring D;
R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
R₈, R₉ and R₁₀ are each independently selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
R₇ and R₈ together represent a C₂ to C₁₀ alkyl, hydroxyalkyl, or alkylene; or
R₇ and R₈ together are: 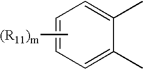
wherein:
m is an integer from 1 to 3, and R₁₁ is selected from one of H and —CONHR₁₂NR₁₃R₁₄, wherein R₁₂ is alkyl and R₁₃ and R₁₄ are each independently selected from one of H and alkyl; or
a pharmaceutically acceptable salt thereof.

100. The pharmaceutical formulation of claim 99, wherein:
X and Y are each CH;
R₃ is selected from the group consisting of H, alkyl, hydroxyl, alkoxyl, and araloxyl;
R₄ is selected from one of H and halogen;
R₅ is H;
Z is: 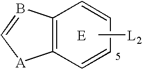
wherein:
A is NH;
B is N;
L₁ and L₂ are each independently: 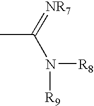
wherein:
R₇ is selected from one of H and hydroxyl; and
R₈ and R₉ are each H.

101. The pharmaceutical formulation of claim 100, wherein R₃ is H.

102. The pharmaceutical formulation of claim 100, wherein R₃ is alkyl.

103. The pharmaceutical formulation of claim 100, wherein R₃ is hydroxyl.

104. The pharmaceutical formulation of claim 100, wherein R₃ is araloxyl.

105. The pharmaceutical formulation of claim 100, wherein R₄ is H.

106. The pharmaceutical formulation of claim 100, wherein R₄ is halogen.

107. The pharmaceutical formulation of claim 100, wherein R₇ is H.

108. The pharmaceutical formulation of claim 100, wherein R₇ is hydroxyl.

109. The pharmaceutical formulation of claim 99, wherein:
X is N;
Y is CH;
R₃, R₄, and R₅ are each H;
Z is: 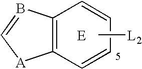
wherein:
A is NH;
B is N;
L₁ and L₂ are each independently: 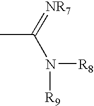
wherein:
L₁ is in the 4′-position of the diaryl ring D;
R₇ is selected from one of H and hydroxyl; and
R₈ and R₉ are each H.

110. The pharmaceutical formulation of claim 109, wherein R₇ is H.

111. The pharmaceutical formulation of claim 109, wherein R₇ is OH.

112. The pharmaceutical formulation of claim 99, wherein:
X and Y are each CH;
R₃, R₄, and R₅ are each H;
Z is: 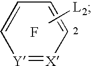
X′ is O;
Y′ is absent;
L₁ and L₂ are each independently selected from the group consisting of: 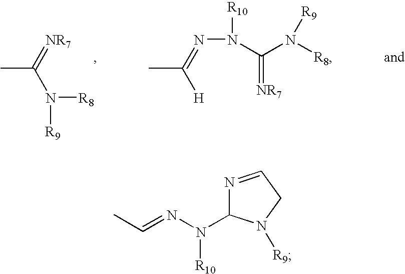
wherein:
R₇ is selected from one of H and OH; and
R₈, R₉ and R₁₀ are H.

113. The pharmaceutical formulation of claim 112, wherein L₁ and L₂ are each independently:

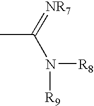

114. The pharmaceutical formulation of claim 113, wherein R₇ is H.

115. The pharmaceutical formulation of claim 113, wherein R₇ is OH.

116. The pharmaceutical formulation of claim 112, wherein L₁ and L₂ are each independently: 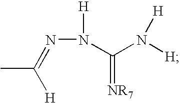
and R₇ is H.

117. The pharmaceutical formulation of claim 112, wherein L₁ and L₂ are each independently:

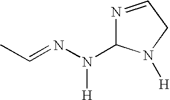

118. The pharmaceutical formulation of claim 99, wherein the compound is selected from the group consisting of:
2-(4′-carbamimidoyl-2′-methylbiphenyl-4-yl)-1H-benzimidazole-5-carboxamidine;
2-[4-(5-carbamimidoyl-pyridin-2-yl)-phenyl]-1H-benzimidazole-5-carboxamidine;
2-(4′-carbamimidoyl-3′-fluoro-biphenyl-4-yl)-1H-benzimidazole-5-carboxamidine; and
2-(4′-carbamimidoyl-2′-hydroxy-biphenyl-4-yl)-1H-benzimidazole-5-carboxamidine.

119. The pharmaceutical formulation of claim 99, wherein the compound is selected from the group of compounds having the following chemical structures:

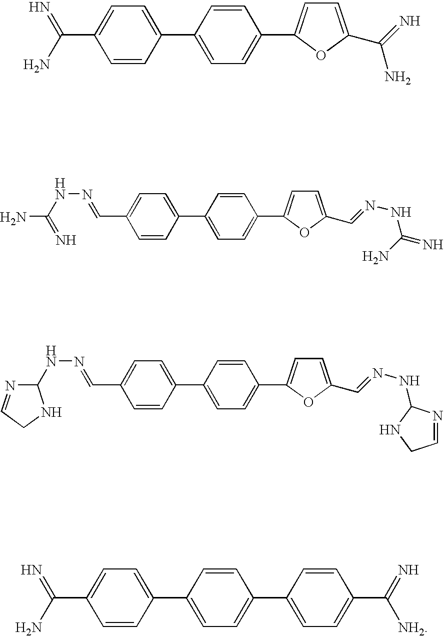

120. The pharmaceutical formulation of claim 99, wherein the compound comprises a pharmaceutically acceptable salt.

121. The pharmaceutically acceptable salt of claim 120, wherein the pharmaceutically acceptable salt comprises an acetate salt.

122. A method of treating microbial infection in a subject in need thereof, the method comprising administering to the subject an effective amount of a compound comprising a diaryl ring structure of Formula (II): 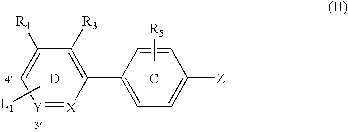
wherein:
X and Y are each independently selected from the group consisting of CH, N, O and S, and Y can be present or absent;
R₃, R₄, and R₅ are each independently selected from the group consisting of H, alkyl, halogen, hydroxyl, alkoxyl, aryloxyl, and aralkoxyl;
Z is selected from one of: 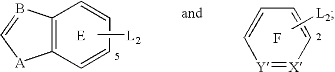
wherein:
A is selected from the group consisting of O, S, and NR₆, and wherein R₆ is selected from one of H and alkyl;
B is selected from the group consisting of O, S, and N;
X′ and Y′ are each independently selected from the group consisting of CH, N, O and S, and Y′ can be present or absent;
L₁ and L₂ are each independently selected from the group consisting of: 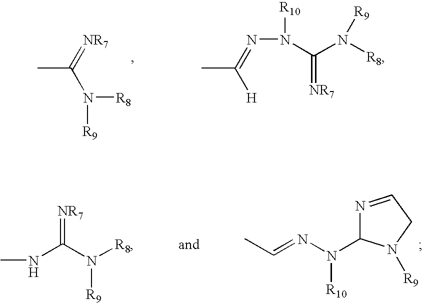
wherein:
L₁ is at one of the 3′-position and the 4′-position of the diaryl ring D;
R₇ is selected from the group consisting of H, alkyl, hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl;
R₈, R₉ and R₁₀ are each independently selected from the group consisting of H, alkyl hydroxyl, alkoxyalkyl, cycloalkyl, aryl, aralkyl, alkoxyl, hydroxylalkyl, hydroxycycloalkyl, alkoxycycloalkyl, acyloxyl, aminoalkyl, and alkylaminoalkyl; or
R₇ and R₈ together represent a C₂ to C₁₀ alkyl, hydroxyalkyl, or alkylene; or
R₇ and R₈ together are: 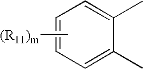
wherein:
m is an integer from 1 to 3, and R₁₁ is selected from one of H and —CONHR₁₂NR₁₃R₁₄, wherein R₁₂ is alkyl and R₁₃ and R₁₄ are each independently selected from one of H and alkyl; or
a pharmaceutically acceptable salt thereof.

123. The method of claim 122, wherein:
X and Y are each CH;
R₃ is selected from the group consisting of H, alkyl, hydroxyl, alkoxyl, and araloxyl;
R₄ is selected from one of H and halogen;
R₅ is H;
Z is: 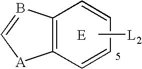
wherein:
A is NH;
B is N;
L₁ and L₂ are each independently: 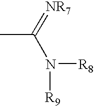
wherein:
R₇ is selected from one of H and hydroxyl; and
R₈ and R₉ are each H.

124. The method of claim 123, wherein R₃ is H.

125. The method of claim 123, wherein R₃ is alkyl.

126. The method of claim 123, wherein R₃ is hydroxyl.

127. The method of claim 123, wherein R₃ is araloxyl.

128. The method of claim 123, wherein R₄ is H.

129. The method of claim 123, wherein R₄ is halogen.

130. The method of claim 123, wherein R₇ is H.

131. The method of claim 123, wherein R₇ is hydroxyl.

132. The method of claim 122, wherein:
X is N;
Y is CH;
R₃, R₄, and R₅ are each H;
Z is: 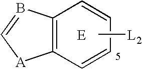
wherein:
A is NH;
B is N;
L₁ and L₂ are each independently: 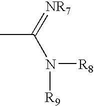
wherein:
L₁ is in the 4′-position of the diaryl ring D;
R₇ is selected from one of H and hydroxyl; and
R₈ and R₉ are each H.

133. The method of claim 132, wherein R₇ is H.

134. The method of claim 132, wherein R₇ is OH.

135. The method of claim 122, wherein:
X and Y are each CH;
R₃, R₄, and R₅ are each H;
Z is: 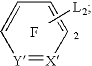
X′ is O;
Y′ is absent;
L₁ and L₂ are each independently selected from the group consisting of: 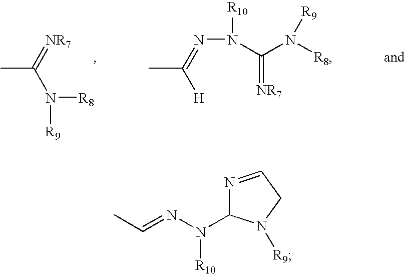
wherein:
R₇ is selected from one of H and OH; and
R₈, R₉ and R₁₀ are H.

136. The method of claim 135, wherein L₁ and L₂ are each independently:

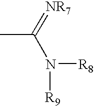

137. The method of claim 136, wherein R₇ is H.

138. The method of claim 136, wherein R₇ is OH.

139. The method of claim 135, wherein L₁ and L₂ are each independently: 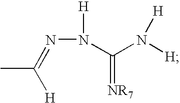
and R₇ is H.

140. The method of claim 135, wherein L₁ and L₂ are each independently:

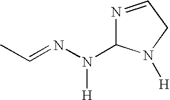

141. The method of claim 122, wherein the compound is selected from the group consisting of:
2-(4′-carbamimidoyl-2′-methylbiphenyl-4-yl)-1H-benzimidazole-5-carboxamidine;
2-[4-(5-carbamimidoyl-pyridin-2-yl)-phenyl]-1H-benzimidazole-5-carboxamidine;
2-(4′-carbamimidoyl-3′-fluoro-biphenyl-4-yl)-1H-benzimidazole-5-carboxamidine; and
2-(4′-carbamimidoyl-2′-hydroxy-biphenyl-4-yl)-1H-benzimidazole-5-carboxamidine.

142. The method of claim 122, wherein the compound is selected from the group of compounds having the following chemical structures:

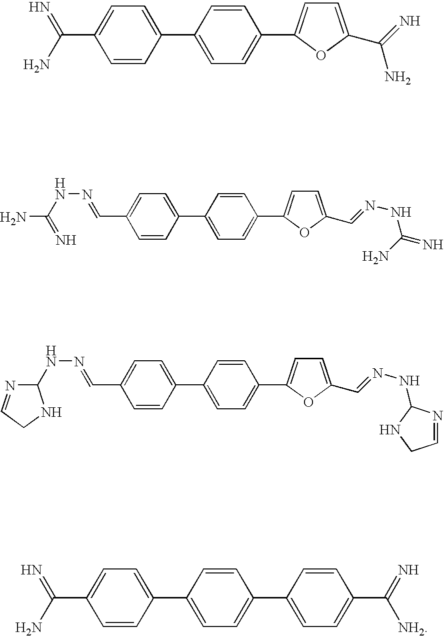

143. The method of claim 122, wherein the compound comprises a pharmaceutically acceptable salt.

144. The pharmaceutically acceptable salt of claim 143, wherein the pharmaceutically acceptable salt comprises an acetate salt.

145. The method of claim 122, wherein the microbial infection is selected from one of a Trypanosoma brucei rhodesiense infection and a Plasmodium falciparum infection.

146. The method of claim 145, wherein the microbial infection comprises a Trypanosoma brucei rhodesiense infection.

147. The method of claim 145, wherein the microbial infection comprises a Plasmodium falciparum infection.
